(19) United States
(12) Patent Application Publication
(10) Pub. No.: US 2006/0263881 A1
(43) Pub. Date: Nov. 23, 2006

(54) Methods of expanding and selecting disease associated T-Cells

(75) Inventors: Keld Kaltoft, Hammel (DK); Jorgen Agnholt, Risskov (DK)

Correspondence Address: CHERYL H AGRIS PHD, PO BOX 806, PELHAM, NY, 10803, US

(73) Assignee: CELLCURE A/S, Kobenhavn SV (DK)

(21) Appl. No.: 11/490,816

(22) Filed: Jul. 21, 2006

Related U.S. Application Data
(63) Continuation of application No. 09/720,371, filed on Apr. 30, 2001.
(60) Provisional application No. 60/091,684, filed on Jul. 2, 1998.

(30) Foreign Application Priority Data
Jun. 26, 1998 (DK) PA 1998 00848
Jul. 1, 1998 (DK) PA 1998 00895

(51) Int. Cl. C12N 5/08

(52) U.S. Cl. 435/366; 435/372

(57) ABSTRACT

Methods of expanding and selecting disease associated T-cells, continuous T-cell lines as well as T-cell lines obtainable by these methods are disclosed. Furthermore, pharmaceutical compositions and vaccines comprising activated disease associated T-cell are disclosed. The uses of the T-cell and T-cell lines are numerous and include methods of diagnosis, methods for the treatment, alleviation or prevention of diseases associated with activation of T-cells, methods of testing the effect of medicaments against T-cell associated diseases, methods of detecting T-cell growth factors, methods of monitoring the response to treatment, alleviation or prevention of diseases associated with activation of T-cells, and methods of identifying disease associated antigens.

The present invention relates to methods of expanding and selecting disease associated T-cells, continuous T-cell lines as well as T-cell lines obtainable by the methods. The invention also relates to pharmaceutical compositions comprising activated disease associated T-cell. In a further aspect, the invention relates to vaccines comprising such activated disease associated inflammatory T-cells. The invention further relates to pharmaceutical compositions for use in adjuvant treatment comprising disease associated regulatory or cytotoxic T-cells. Furthermore, the present invention concerns the use of T-cell lines for preparing medicaments for treating T-cell associated diseases as well as for use in a broad range of methods, i.a. methods of diagnosis, methods for the treatment, alleviation or prevention of diseases associated with activation of T-cells, methods of testing the effect of medicaments against T-cell associated diseases, methods of detecting T-cell growth factors, methods of monitoring the response to treatment, alleviation or prevention of diseases associated with activation of T-cells, and methods of identifying disease associated antigens. The present invention also concerns a model system for testing the effect of a medicament against a T-cell associated disease.

### BACKGROUND OF THE INVENTION

All normal somatic cells are believed to have a finite in vitro life-span commonly known as the Hayflick limit. This dogma is a cornerstone in cell biology. According to this, only a certain number of cell population doublings (PD) is possible. Following approximately 23 PD, T-cells go into replicative senescence, and the cells cease to divide. This implies that one T-cell can on average expand only to 2²³ cells corresponding to approximately 10⁷ T-lymphocytes. Most often 10⁷ T-lymphocytes, that is about 10 mg, are not enough “material” for use as T-cell vaccine in treatment of patients with T-cell-related auto-immune/chronic inflammatory diseases or for the use as T-cell adjuvant therapy in patients with inflammatory/auto-immune or malignant diseases. By way of example, in cancer 10 mg of clonal cytotoxic T-cells is far to little to combat tumour masses n the order of kilograms.

In the prior art, there is several examples of attempts to overcome this problem. However, none has come up with the solution presented in the present invention. Several publications relate to activated T-cells wherein antigen specific T-cells are produced ex vivo after stimulation in vitro with a known antigen. The T-cells are commonly produced from peripheral blood T-cells by procedures, in which an antigen is used to stimulate T-cells. The antigen specific T-cell clones are obtained by using conventional immunological selection techniques. Only a few successful attempts to produce disease-associated T-cells in sufficient amount have been reported.

WO 88/07077 (Liu) (ref. 1) discloses a method of expanding helper T-cells (Tₕ-cells) recognising viral antigens, wherein Tₕ cells are made to proliferate from a sample of mononuclear cells including the Tₕ-cells and antigen presenting cells (APCs) by the addition of specific viral antigen. The proliferating Tₕ-cells may be expanded in the presence of APCs and specific antigen. Optionally IL-2 may be added in order to stimulate the expansion.

WO 94/02156 (Engelman) (ref. 2) discloses a method of activating T-cell isolated from peripheral blood, wherein specific antigen is used to pulse dendritic cells and thereafter mixed with the isolated T-cells. The mixture is expanded in the presence of IL-2 and/or IL-4, however, in very low concentrations (about 2 IU/ml).

WO 97/05239 (Gruenberg) (ref. 3) discloses a method of expanding T-cells isolated from the peripheral blood, wherein the expansion is performed without IL-2 due to its alleged toxic effect in humans.

Kaltoft et al. showed in 1995 (ref. 4) that continuous T-lymphocyte cell lines can be established from chronic inflammatory skin diseases, when the culture medium is supplemented with IL-2 and IL-4, but without antigen and accessory cells added. These cell lines have been shown by far to exceed the Hayflick limit. However, the authors did not realise that what they observed was a way of expanding antigen specific disease associated T-cells in unlimited quantities. Among the theories concerning the immortalised T-cell lines disclosed by Kaltoft et al. (1995) (ref. 4), the following were suggested: Chromosome abnormalities, faulty selection in thymus, induction by virus, effect of the inflammation itself, loss of the T-cell antigen receptor complex or other intrinsic factors as discussed in the article. This is also supported in the subsequent review of the subject (Effros et al.) (ref. 5), wherein the chromosomal abnormalities are mentioned as the relevant thesis for escape from the replicative senescence of the T-cells.

Human T-cell vaccination has been known since 1988. The principle is based on the hypothesis that auto-immune diseases like disseminated sclerosis, rheumatoid arthritis and Crohn's disease are caused by antigen associated/specific T cells participating in a regulatory network. The activity of inflammatory T-cells (IFNγ and TNFα producing) is regulated by IL-10 producing regulatory T cells (In a type 1 inflammatory process, the inverse in type 2 inflammatory processes), cf. FIG. 1.

In human studies, it has been very difficult to obtain the relevant auto-reactive T-cells and propagate these cells into sufficient amounts to produce T-cell vaccines, although T-cell vaccination studies in disseminated sclerosis has been promising.

Surprisingly, it has now been recognised that continuous T-cell lines are obtainable by a method of expanding and selecting disease associated T-cells. The principle of the present invention is based on in vivo antigen stimulation, this in vivo stimulation leading to the presence of a certain population of activated T-cells, and this T-cell population can be expanded and selected under certain conditions. T-cells associated with the manifestations of a disease are activated in vivo, and, may therefore often be expanded in vitro without further supplement of a disease associated antigen. Furthermore, the T-cells are activated in vivo in such a manner that they are able to grow in vitro under special conditions. No cloning step is necessary. The activated T-cells are ready to expand and may therefore outgrow non-activated T-cells. The pool of activated T-cells in a biopsy can contain T-cells with different specificities and functions as well as being of different phenotypes. Selection of a T-cell line with a desired phenotype, specificity and function may be controlled by the conditions of the growth media, and by immunoselection methods.

### SUMMARY OF THE INVENTION

Thus, in the broadest aspect, the present invention relates to a method of expanding and selecting disease associated T-cells, which method comprises
(a) obtaining a tissue sample from a mammal including a human being, the sample comprising disease activated T-cells, or
   obtaining T-cells and antigen-presenting cell from said mammal and mixing said cells with a disease associated antigen or antigens, and
(b) culturing said tissue sample or said mixture of cells and antigen(s) in the presence of at least two factors which promote T-cell growth and optionally one or more additional compounds.

In a further aspect, the present invention relates to such continuous T-cell lines obtainable by the method.

The uses of the disease associated T-cells prepared according to the method, or the T-cell lines obtainable by the method are numerous. In particular, the T-cells and T-cell lines may be used as the active ingredient in pharmaceutical compositions and vaccines. The T-cells or T-cell lines may further be used for preparing a medicament for the treatment of various T-cell associated diseases, including diseases of inflammatory, auto-immune, allergic, neoplastic, or transplantation-related origin, or combinations thereof.

Furthermore, the T-cells or the T-cell lines can be used in methods for diagnosing diseases, methods for treating, alleviating or preventing diseases associated with activation of T-cells, methods of testing the effect of a medicament against a T-cell associated disease, methods for the treatment, alleviation or prevention of diseases associated with T-cell activation, methods of detecting T-cell growth factors, methods of monitoring the effect of or response to treatment against T-cell associated diseases including diseases of inflammatory, auto-immune, allergic, neoplastic or transplantation-related origin or combinations thereof, and methods of identifying disease associated antigens.

Model systems for testing the effect of a medicament against T-cell associated diseases also forms part of the invention.

### BRIEF DESCRIPTION OF THE FIGURES

FIG. 1 shows schematically the T-cell vaccination principle.

FIG. 2 shows schematically the establishment of a T-cell culture.

FIG. 3 shows schematically the T-cell vaccination procedure.

FIG. 4. Shows the number of cell population doublings, D, of three PBMC cultures grown in medium with IL-2+IL-4 alone (left) or with allostimulation in the presence of IL-2+IL-4 (right).

FIG. 5. Shows telomerase activity at 100 PD of a continuous peripheral blood activated CD4+ cell line (Act-1) cultured with IL-2+IL-4, IL-2 or IL-4 as indicated. For comparison, telomerase activity of the leukemic cell line Se-Ax, cultured with IL-2 alone, is also shown.

FIG. 6. Shows CD28 expression of the continuous peripheral blood derived CD4+ cell line Act-1 at PD 60 and 150 compared with CD4 and CD8 expression at PD 150 (Flow cytometric analysis).

FIG. 7. Shows CD28 expression at different PD of the clonal T-cell line My-La, 46,XY,i(18q). Also shown is CD4 and Vₚ18 expression at the different PD and CD8 expression at PD 200 (Flow cytometric analysis)

FIG. 8. Shows the phenotype in the growing primary T-cell culture from which Gut_{I}-1 is derived (Example 2) (Flow cytometric analysis).

FIG. 9. Shows the phenotype of Gut_{R}-2 in Example 2 (Flow cytometric analysis).

FIG. 10. Shows the karyotype 45,XY_{I},-20,add(1) (p36) of Gut_{R}-2.

FIG. 11. Shows the phenotype in the growing continuous T-cell culture of Gut_{I}-1 (Example 2) (Flow cytometric analysis).

FIG. 12. Shows the karyotype 47,XX_{I},+2,t(1;1) of Gut_{I}-1.

FIG. 13. FACS analysis of transmembrane TNFα in the four primary cultures. Two of the primary cultures were stimulated with super-antigen (SEA: Staphylococcus enterotoxin A). Lines indicate determination without Infliximab, and red line supplement of Infliximab to the cultures, respectively.

FIG. 14 FACS analysis of transmembrane TNFα. Stimulation of three long term cultured cultures with super-antigen. Lines indicate determination without Infliximab and supplement of Infliximab to the cultures, respectively. (SEA: Staphylococcus enterotoxin A)

FIG. 15. INFγ production in primary cultures before and after supplement with Infliximab.

FIG. 16. INFγ and TNFβ production in primary culture C8.3 before and after stimulation with super-antigen. (Ifx: Infliximab; SEA: Staphylococcus enterotoxin A).

FIG. 17. INFγ and TNFα production in long term cultured cultures C1x, C2x, C4.2 before and after stimulation with super-antigen. (SEA: Staphylococcus enterotoxin A).

FIG. 18. Detection of apoptosis by Annexin FITC and propidium iodide (PI). Cells in apoptosis: FITC positive and located in lower right quadrant (LR). Cells in necrosis are double positive (FITC and PI positive and located in upper right quadrant). Negative cells located in lower left quadrant. (Ifx: Infliximab; SEA: Staphylococcus enterotoxin A, C3: complement, KL II: class II antibody (L 243 mouse anti human (Becton Dickinson)).

FIG. 19 A-C. Coulter counter particle count with analysis of viable cells between cursor statistics.

FIG. 20 A and B. In this figure, melanoma cells alone are shown (FIG. 20A). Furthermore, melanoma cells and cytotoxic T-cells are shown. FIG. 20B shows that melanoma cells are eliminated within 24 hours, leaving only some T-lymphocytes in the culture.

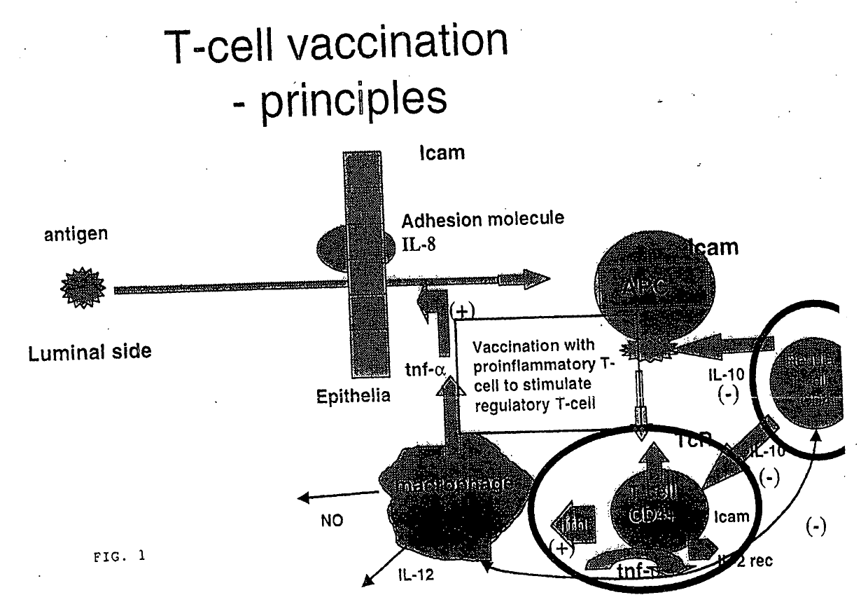

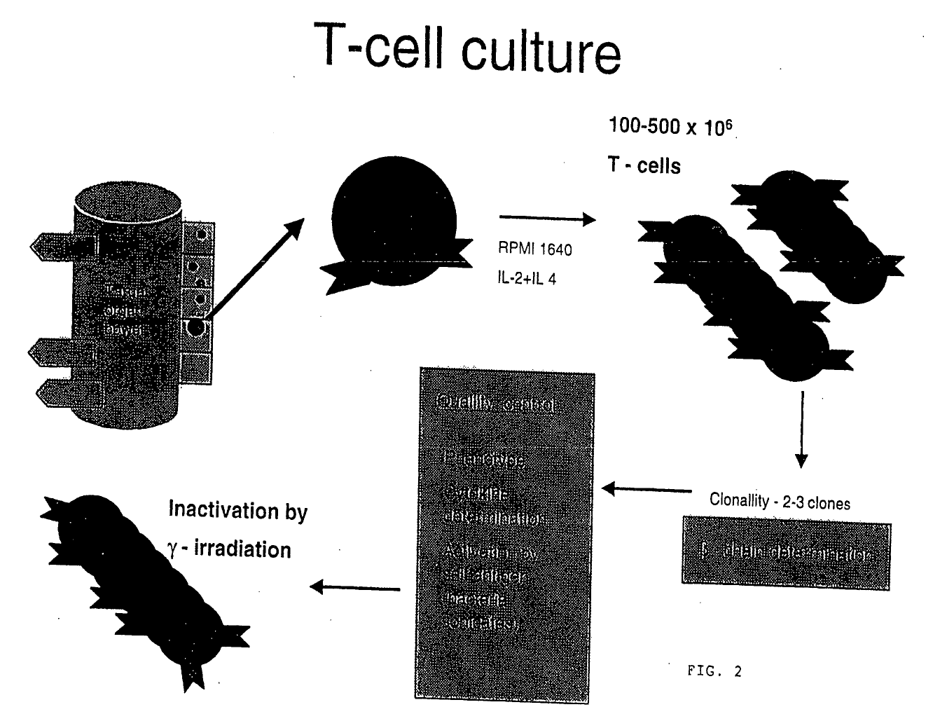

### DETAILED DESCRIPTION OF THE INVENTION

As mentioned above, the present invention is based on the recognition that certain T-cells which are associated with diseases may be expanded selectively. Such T-cells have been stimulated in vivo by a disease associated antigen or antigens. Surprisingly, such T-cells can be expanded and selected in vitro under certain conditions, whereby the T-cells escape replicative senescence and become continuous or immortal. Surprisingly, the T-cell lines maintain their antigen specificity and function during continuous culturing.

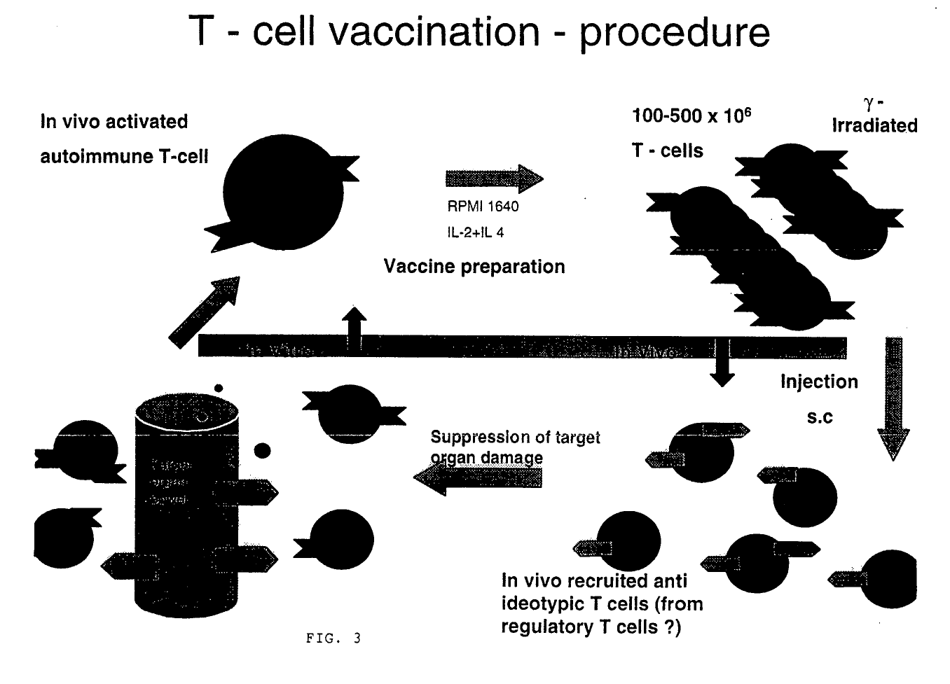

In the present invention, a cell culture system is introduced where the relevant T-cell can be expanded in practically unlimited amounts. By a quality control system (FIG. 2), it will be possible to produce T-cells which could be relevant for T-cell vaccination treatment (alternatively adjuvant treatment) in for example Crohn's disease.

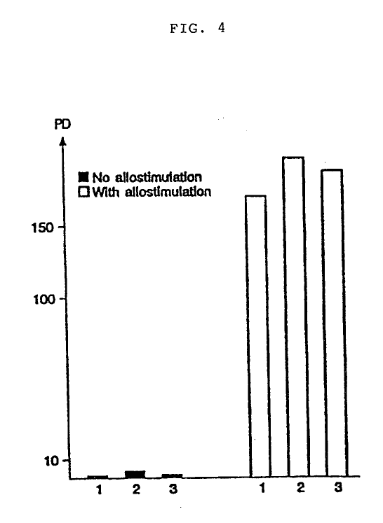

In Crohn's disease the principle is based on the fact that the disease is associated with increased activity in type 1 inflammatory T-cells (IFNγ and TNFα cells. The activity is not sufficient to activate the regulatory T-cells (IL-10 producing), but is sufficient to induce proteolytic degradation of the intestinal mucousa (active disease). In a T-cell vaccination the regulatory T-cell activity will be increased by boosting the activity by injection of attenuated activated inflammatory T-cells expanded into sufficient amounts (FIG. 3).

“Continuous” or “immortal” is intended to mean that the cells have a life-span of at least 40 PD (i.e. 1 cell becoming approximately 1 kg of cell mass), such as at least 60 PD (i.e. 1 cell becoming approximately 100 tons of cell mass), preferably at least 100 PD, more preferably at least 150 PD, such as at least 200 PD. It is further preferred that the functional profile of the T-cells are not substantially altered during the continuous growth meaning that the function of the T-cells essentially correspond to the initial cells. In certain cases, re-activation with antigen, antibodies or chemical compounds may be used to activate the T-cells to an increased growth rate. The final aim of the invention is that an unlimited amount of specific T-cells may be produced.

The method of expanding and selecting disease associated T-cells of the invention comprises
(a) obtaining a tissue sample from a mammal including a human being, the sample comprising disease activated T-cells, or
   obtaining T-cells and antigen-presenting cell from said mammal and mixing said cells with a disease associated antigen or antigens, and
(b) culturing said tissue sample or said mixture of cells and antigen(s) in the presence of at least two factors which promote T-cell growth and optionally one or more additional compounds.

“Disease associated T-cells” are intended to comprise all T-lymphocytes present at the site of disease.

By the term “disease activated T-cells” is meant the fraction of disease associated T-cells that are activated by the inflammatory process taking place at the site of disease.

In the present context, the expressions “T-cell” and “T-lymphocyte” are used interchangeably.

The term “disease associated antigen(s)” is intended to comprise antigen(s) (foreign or auto-antigen(s)) that initiate and maintains the inflammatory response.

By the term “factors which promote T-cell growth” is meant biological and/or chemical compounds, cells and the like which directly and/or indirectly stimulate T-cell growth.

The activated disease associated T-cells can be obtained in a tissue sample comprising such cells, which sample is taken from a mammal including a human being. Alternatively, the disease associated T-cells can be derived by obtaining T-cells and antigen presenting cells (APCs) from a mammal including a human being, and mixing such cells with a disease associated antigen or antigens. The T-cells may originate from a mammal being inflicted with a T-cell associated disease or from a healthy mammal. In particular, the tissue sample is a biopsy taken at the site of the disease. Such tissue sample is expected to further comprise antigen presenting cells as well as the antigen(s) that caused the activation of the T-cells.

Factors which promote T-cell growth may be selected from the group consisting of cytokines which promote T-cell growth. Examples of such cytokines are IL-2, IL-15, IL-4, IL-7, IL-9, IL-10, IL-16, and functionally similar cytokines. In particular, a combination of (1) IL-2 and/or IL-15, and (2) IL-4 and/or IL-7 and/or IL-9 may be used. In one embodiment of the present method, a combination of IL-2 and IL-4 is used. However, other T-cell growth promoting factors may also be used. Examples are combinations of ligation of the surface markers CD2, CD3 or CD28 with antibodies directed against CD2, CD3 or CD28.

By the term “functionally similar” is meant that the effect observed are comparable to the effect observed by the cytokines mentioned in the context of the present invention. These functionally similar compounds may substitute the specifically mentioned compounds in the specific process referred to.

The cytokines are preferably used in a concentration of at least 1 nM each, preferably more than 2.5 nM, more preferably than 10 nM each. The concentration of the cytokines might not be important, however, the concentration should be chosen so as to ensure growth, i.e. at least 1 nM of each. Traditionally, the concentration of a cytokine is expressed as activity in units per ml (u/ml). The person skilled in the art will readily know how to interrelate u/ml and concentration (molar, M). If nothing else is stated, it is to be assumed that 200 u/ml equals 1 nM.

The T-cells and APCs are preferably obtained from any body fluid including peripheral blood, and further from the spleen, the lymph nodes and thymus, and by spinal puncture.

The T-cells to be cultured originates preferably from a tissue sample. The tissue sample is preferably selected from a biopsy, from sputum, swaps, gastric lavage, bronchial lavage, and intestinal lavage, or body fluids such as spinal, pleural, pericardial, synovial, blood and bone marrow.

A biopsy can in principle be taken from any organ including the pancreas, the intestines, the liver, the kidneys, the lymph nodes, the breasts, and from the skin. Furthermore, peripheral blood may also be a suitable source of T-cells. Preferably the cells are taken from the organ of the disease.

In one embodiment of the present method, the disease associated T-cells are CD4+, CD8+ or CD4−/CD8− T-cells.

In particular, the disease associated T-cells are inflammatory, cytotoxic or regulatory T-cells.

Within the present context “inflammation” is defined as a general term for the local accumulation of fluid, plasma proteins, and white blood cells that is initiated by physical injury, infection, or a local immune response. This is also known as a inflammatory response. Acute inflammation is the term used to describe transient episodes, whereas chronic inflammation occurs when the infection persists or during auto-immune responses. Many different forms of inflammation are seen in different diseases. The cells that invade tissues undergoing inflammatory responses are often called inflammatory cells or an inflammatory infiltrate.

The majority of chronic inflammatory/auto-immune disease fall within two major groups: A type 1 chronic inflammation dominated by production of primarily IFNγ and TNFα (a type 1 inflammatory cytokine profile) or a type 2 chronic inflammation dominated by production of primarily IL-4 and IL-5 (a type 2 cytokine production). Examples of type 1 chronic inflammatory/auto-immune disease are multiple sclerosis and Crohn's disease, whereas examples of type 2 chronic inflammatory diseases are asthma and long-standing severe atopic dermatitis.

As IL-4 down-regulates the production of IFNγ, lymphocytes producing IL-4 down-regulate disease activity of a type 1 chronic inflammatory disease through an interactive cellular network. IL-4 producing T cells can thus be considered regulatory T cells in a type 1 chronic inflammatory disease, implicating that in chronic inflammatory disease type 1, the balance between cells producing type 1 cytokines like IFNγ and TNFα are not sufficiently controlled by opposing regulatory T cells producing IL-4.

Conversely, as IFNγ down-regulates production of IL-4, lymphocytes producing IFNγ down-regulate disease activity of a type 2 chronic inflammatory disease through an interactive network. IFNγ producing T-cells are thus considered regulatory T cells in a type 2 chronic inflammation/auto-immune reaction. In type 2 auto-immune/inflammatory disease, the type 2 cytokine production is not sufficiently controlled by opposing IFNγ producing regulatory T-cells.

As IL-10 and TGFβ producing T-cells down-regulate chronic inflammation of both type 1 and type 2, Il-10 and TGFβ producing T-cells are for both types of chronic inflammatory diseases considered to be regulatory.

The definition of inflammatory and regulatory T-cells is thus a relative term depending on the type (type 1 or type 2) of inflammation. In type 1 chronic inflammation, the T-cells producing type 1 cytokines are considered inflammatory T-cells, and IL-4 or IL-10 and TGFβ producing T-cells are considered regulatory T-cells.

If the chronic inflammation is dominated by type 2 cytokines, the type 2 cytokine producing T-cells are considered inflammatory T-cells, whereas IFNγ and/or IL-10 producing T-cells are considered regulatory in this type of disease.

Preferably, the disease associated T-cells are associated with a disease of inflammatory, auto-immune, allergic, neoplastic or transplantation-related origin, or combinations thereof. In particular, the disease of inflammatory or allergic origin is a chronic inflammatory disease or a chronic allergic disease.

Diseases of inflammatory/auto-immune origin include asthma, hypersensitivity pneumonitis, interstitial lung disease, sarcoidosis, idiopathic pulmonary fibrosis, interstitial lung disease associated with Crohn's Disease or ulcerative colitis or Whipple's disease, interstitial lung disease associated with Wegeners granulomatosis or hypersensitivity vasculitis,

vasculitis syndromes, Hennoch-Schönleins purpura, Goodpastures syndrome, Wegeners granulomatosis,

renal diseases such as antibody mediated glomerulopathia as in acute glomerulonephritis, nephritis associated with systemic lupus erythematosus, nephritis associated with other systemic diseases such as Wegeners granulomatosis and Goodpastures syndrome and mixed connective tissue disease, chronic interstitial nephritis, chronic glomerulonephritis,

gastrointestinal diseases such as Crohn's Disease, Ulcerative colitis, coeliac disease, Whipple's disease, collagenous colitis, eosinophillic colitis, lymphatic colitis,

hepatobilliary diseases such as auto-immune hepatitis, alcohol induced hepatitis, periportal fibrosis, primary billiary cirrhosis, sclerosing colangitis,

disorders of the central or peripheral nervous system such as demyelinating disease as multiple sclerosis, acute disseminated encephalomyelitis, sub-acute sclerosing panencephalitis,

skin disease such as psoriasis, atopic dermatitis, eczema, allergic skin disease, progressive systemic sclerosis (scleroderma), exfoliating dermatitis, pemphigus vulgaris,

joint diseases such as rheumatoid arthritis, ankylosing spondylitis, arthritis associated with psoriasis or inflammatory bowel disease,

muscoloskelletal diseases such as myastenia gravis, polymyositis,

endocrine diseases such as insulin dependent diabetes mellitus, auto-immune thyroiditis (Hashimoto), thyreotoxicosis, Graves,

diseases of the hematopoetic system such as auto-immune anaemia, auto-immune thrombocytopenia,

cardiovascular diseases such as cardiomyopathia, vasculitis, cardiovascular disease associated with systemic diseases as systemic lupus erythematosus, polyarthritis nodosa, rheumatoid arthritis, scleroderma, sarcoidosis.

Diseases of neoplastic origin include malignant melanoma, Sezary's syndrome, cutaneous T-cell lymphoma, renal cell carcinoma, colorectal cancer, breast cancer, ovarian cancer, cancer of the uterus, prostatic cancer, hepatic carcinoma, lung cancer, and sarcoma.

Furthermore, disorders relating to transplantation may be disorders which can be treated, alleviated or prevented by use of the method of the present invention.

Chronic rejection may be related to the development of pro-inflammatory type 1 cytokine producing T-cells, and, accordingly, the expansion and selection of regulatory T-cells for adjuvant treatment in such patients may be of relevance.

In a particular embodiment of the present invention, the disease is an inflammatory bowel disease, Crohn's disease, ulcerative colitis, sclerosis, type I diabetes, rheumatoid arthritis, psoriasis, atopic dermatitis, asthma, malignant melanoma, renal carcinoma, breast cancer, lung cancer, cancer of the uterus, prostatic cancer, hepatic carcinoma, or cutaneous lymphoma.

The disease associated T-cells are preferably CD4+ (positive), CD8+, or CD4-(negative)/CD8− T-cells. The disease associated T-cells are suitably, according to the definition of inflammation, such which are inflammatory T-cells or regulatory T-cells. In one embodiment, the regulatory T-cells are cytotoxic T-cells, or CD4+ T-cells which in the case of a type 1 inflammation produce IL-4 or IL-10 and TGFβ, or in the case of a type 2 inflammation produce INFγ or IL-10 and TGFβ. In another embodiment, the inflammatory T-cells are T-cells involved in chronic inflammatory/auto-immune diseases falling within the two major groups: A type 1 chronic inflammation dominated by production of primarily IFNγ and TNFα (a type 1 inflammatory cytokine profile) or a type 2 chronic inflammation dominated by production of primarily IL-4 and IL-5 (a type 2 cytokine production) Examples of type 1 chronic inflammatory/auto-immune disease are multiple sclerosis and Crohn's disease, whereas examples of type 2 chronic inflammatory diseases are asthma and long-standing severe atopic dermatitis.

In accordance with the present invention, the cells to be expanded and selected may optionally be cultured in the presence of at least two factors which promote T-cell growth and one or more additional compounds which preferably are such as to directly or indirectly interfere with T-cell growth, in particular such which enhance or inhibit growth of inflammatory, regulatory or cytotoxic T-cells. The function of the additional compound is to promote the selection and expansion of a desired function of the T-cells (i.e. inflammatory or regulatory). When such additional compound or compounds is used, it may preferably be selected from cyclosporin, GM-CSF, Prednisone, Tacrolimus, FK506, IL-10, IL-10 antibody, TNFα antibody, IL-12, anti-IL-12, IL-7, anti-IL-7, IL-9, anti-IL-9, IL-16, caspase inhibitors, and similar compounds.

In another embodiment, the method comprises a selection procedure. Such selection procedure is described in further detail below.

Inflammatory cells may suitably be cells having a CD4+ phenotype and a type 1 cytokine profile. The inflammatory T-cells are in particular cells contributing in a type 1 inflammatory infiltrate, which cells further produce INFγ and TNFα.

As mentioned above, the selection is accomplished by addition of one or more additional compounds selected from cyclosporine, Prednisone, Tacrolimus, FK506, GM-CSF, IL-12, IL-16, anti-IL-10, anti-TNFα, and functionally similar compounds.

In another aspect of the present method, the inflammatory T-cells are cells having a CD4+ phenotype and a type 2 cytokine profile. Such inflammatory T-cells are in particular cells contributing in a type 2 inflammatory infiltrate, which cells produce IL-4 and IL-5.

As mentioned above, the selection is accomplished by addition of one or more additional compounds selected from cyclosporin, Prednisone, Tacrolimus, FK506, GM-CSF, IL-16, anti-IL-12, and functionally similar compounds.

Thus, the present invention relates to a method as described above, wherein the disease is mediated or partially mediated by type 1 or type 2 inflammatory T-cells such as chronic inflammatory bowel diseases e.g. Crohn's disease and ulcerative colitis, sclerosis, type I diabetes, rheumatoid arthritis, psoriasis, atopic dermatitis, asthma, and transplantation-related diseases.

In another aspect of the present method, disease associated regulatory T-cells are expanded and selected. Such regulatory T-cells are suitably cells having a CD4+ phenotype and a type 1 cytokine profile regulating a type 2 inflammatory disease. In particular, such cells are producing INFγ and/or IL-10. Selection of such T-cells is accomplished by addition of one or more additional compounds selected from IL-10, IL-12 and functionally similar compounds. The invention further relates to a method as described above, wherein the disease is mediated or partly mediated by type 2 inflammatory T-cells, e.g. asthma or atopic dermatitis.

The regulatory T-cells may also be cells having a CD4+ phenotype and a type 2 cytokine profile regulating a type 1 inflammatory disease. Such regulatory T-cells are cells producing IL-10 and/or IL-4. Selection of such regulatory T-cells is accomplished by addition of one or more additional compounds selected from anti-IL-12, IL-10, GM-CSF, IL-16, and functionally similar compounds. Thus, the present invention relates to a method as described above, wherein the disease is mediated or partially mediated by type 1 inflammatory T-cells e.g. chronic inflammatory bowel diseases such as Crohn's disease and ulcerative colitis, sclerosis, type I diabetes, rheumatoid arthritis, and psoriasis.

Furthermore, the present invention relates to a method as described above, wherein disease associated cytotoxic T-cells are expanded and selected. In particular, such cytotoxic T-cells may have a CD8+ phenotype. The cytotoxic T-cells are further preferably tumour infiltrating lymphocytes (TIL) or cells having similar properties. The CD8+ cells are often auto-immune cells that kill tumour cells. The selection of such cells are accomplished by addition of one of more additional compounds selected from GM-CSF, caspase inhibitors such as Z-VAD, α-CD95, IL-10, IL-12, IL-16, and functionally similar compounds. The present invention relates to a method as described above, wherein the disease is of neoplastic origin such as malignant melanoma, renal carcinoma, breast cancer, lung cancer, cancer of the uterus, prostatic cancer, hepatic carcinoma, and cutaneous lymphoma.

In a further aspect, the present invention relates to continuous T-cell lines obtainable by the methods as defined above and claimed herein. In particular, the T-cell line is such, wherein the T-cells are inflammatory T-cells, regulatory T-cells or cytotoxic T-cells.

As demonstrated in Example 1, the antigen specific T-cells overgrow T-lymphocytes not having the desired specificity. It should be noted that in the examples shown, the T-cells with the shortest PD-time (i.e. the fastest growing T-cells) would preferentially be expanded. In general, it is not to be expected that T-lymphocytes with a desired specificity, avidity, growth potential, phenotype and function preferentially expand over T-cells with other antigen specificities. However, the realisation that antigen specific T-cells can be obtained in an unlimited number implies that appropriate selection procedures will be able to establish T-lymphocyte cell lines with the desired specificity, avidity, growth potential, phenotype and function.

As discussed above, in many chronic diseases, the natural balance between inflammatory and regulatory T-cells has been disrupted and cannot find it's way back in balance. For each such disease, it would be possible to select for and expand either inflammatory T-cells or regulatory T-cells. Because of the in vivo activation of the T-cells, the selected and expanded T-cells are antigen-specific, and thus disease-specific. Dependent on the desired route of treatment, the selection of inflammatory T-cells (T-cell vaccination) or regulatory T-cells (adjuvant treatment) may be directed.

Selection for antigen specific T-cell growth is initiated by antigen presentation. In case of a biopsy harbouring disease associated lymphocytes, it is assumed that the biopsy initially, besides the disease associated T-lymphocytes, also contains antigen and antigen presenting cells. Upon expansion of T-lymphocytes, the initial activation by antigen may not be sufficient for continuous T-lymphocyte growth, and in vitro activation of the desired T-lymphocytes may be necessary. In vitro activation requires access to autologous or HLA matched antigen presenting cells. These can be obtained from a blood sample, as so-called mononuclear cells. Furthermore, powerful antigen presenting cells (dendritic cells) can be obtained from mononuclear cells by culturing plastic adherent mononuclear cells in a medium supplemented with granulocyte-macrophage colony stimulating factor (GM-CSF) and IL-4 (both at a concentration above 1000 u/ml). Dendritic cells will develop within 8-20 days.

Having obtained antigen presenting dendritic cells and disease associated T-lymphocytes, a preferential growth advantage of antigen specific T-lymphocytes is to be expected by mixing antigen, dendritic cells and disease associated T-lymphocytes, or peripheral autologous mononuclear cells, as a source of T-lymphocytes in case a biopsy from the diseased organ is not available. The medium should at least contain two factors promoting T-cell growth and an additional factor, the latter to secure transient growth and differentiation of dendritic cells in cases dendritic cells are necessary. A combination of such factors could be the following cytokines: IL-2, IL-4 and GM-CSF. Furthermore, human serum is preferred in order to minimise the autologous mixed leukocyte reaction.

However, antigen activation of T-lymphocytes may lead to proliferation as well as to activation induced cell death (AICD). The balance between proliferation and cell death determines the growth rate (positive or negative) of a cell culture. In order to down-regulate AICD, inhibitors of AICD can be included in the growth medium. Examples of such inhibitors are caspase inhibitors (like Z-VAD) and certain antibodies with reactivity to CD95 (Fas) that prevents Fas-FasL induced cell death. In addition antigen activation of T-lymphocytes may lead to development of T-cells not having the desired phenotype and/or function, implying how further selection and/or counter-selection procedures can be carried out in order to obtain continuous T-lymphocyte cell lines with the desired properties (cf. below).

As the cell culture system promotes the expansion of the fastest growing T-cell clones, bystander cells not having the desired specificity may overgrow the ones having the wanted specificity, reactivity, phenotype and function. As T-lymphocyte growth in general is dependent on IL-2 as well as IL-4, growth cessation may be obtained by withdrawal of one or both of these cytokines. Specific antigen activation of growth arrested T-lymphocyte cell lines is expected to favour proliferation of antigen specific T-cells in a medium with at least two cytokines.

It is important to monitor activation of the T-lymphocytes, as this shows whether the antigen activation is successful, and gives additional information concerning selection/counter-selection of the desired T-lymphocyte sub-population.

Several assays are available to monitor T-cell activation. Activation markers induced on the surface of the T-lymphocytes by antigen activation, such as CD25, CD69 and membrane bound TNFβ may be used to measure the degree of activation, and may also be used by immuno-separation techniques to select for antigen activated T-lymphocytes. Similarly, differentiation markers such as CD4 and CD8 may be used by immuno-separation techniques to select for T-cells with the appropriate phenotype.

Furthermore, selection of sub-populations of T-lymphocytes expressing particular V_{α} and V_{β} subfamilies of the T-cell receptor complex may be very useful. Importantly, if the antigenic peptides bound to the major histocompatibility complex (MHC) are known, peptide-MHC tetramers can be used to immuno-select T-lymphocytes with the desired specificity and avidity.

Effector functions like cytokine production and cell killing gives information regarding the strength of the antigen activation. However, antigen activation of a given sub-population may activate the immunological network given rise to the outgrowth of regulatory T-cells capable of down-regulating the desired sub-population of T-lymphocytes. As an example of this phenomenon, it is believed that in Crohn's Disease the balance between inflammatory T-lymphocytes producing IFNγ and TNFα and regulatory T-cells mainly producing IL-10 has shifted towards the inflammatory T-lymphocytes. However, a powerful activation and expansion of clonal inflammatory T-lymphocytes is expected to be followed by activation and expansion of regulatory T-lymphocytes, which participate in a down regulation of the inflammatory response. In this way the T-cell vaccination with activated and attenuated inflammatory T-lymphocytes results in a down regulation of the disease related elevated level of inflammatory T-lymphocytes. In this case in order to minimise the establishment of regulatory T lymphocytes, addition of cyclosporin A or glucocorticoids, that partially inhibits the inflammatory response, may be useful. In addition, as IL-10 is of importance for establishment of regulatory CD4+ T-cells, neutralising antibodies to IL-10 may be added to the medium. Conversely, if regulatory lymphocytes are to be established, inflammatory T-lymphocytes should be highly activated, and/or IL-10 added to the medium containing at least one additional cytokine.

Apart from antigen activation, other non-specific methods are available that promote T-cell growth, and if combined with appropriate selection procedures as outlined above, may enhance T-lymphocyte growth, in cases where the cell population doubling time is considered too long. Such methods include activation by super-antigen pulsed antigen presenting cells, activation by mitogens (like PHA and jacalin) in the presence of feeder cells or antigen presenting cells, activation by antibodies against CD2, CD3 and CD28, activation by ionomycin and phorbol ester and in case of cross-reactivity with alloantigen, allostimulation with appropriate allogenic cells with or without autologous dendritic cells (the latter possibility in order to obtain cross-priming). AICD can in all the cases mentioned above be blocked by caspase inhibitors.

The principles outlined above are also applicable if cloned T-cells with a given specificity are available.

The disease determines the subtypes of T-cells which could be relevant as a treatment principle. In auto-immune disease T-cell vaccination with a disease antigen, associated pro-inflammatory type 1 cytokine profile (IFNγ and TNFα) T-cell line could be relevant. If it is not possible to select the disease associated antigen reactive pro-inflammatory T-cell line, it may be possible to select a regulatory T-cell line with a type 2 cytokine profile (IL-4/IL-10) which, in an analogous fashion, can be used as a immunoadjuvant therapy against the disease associated inflammatory T-cells.

In order to select for the desired type of T-cell immunological selection principles or additional compounds can be used as described above.

In the following, a selection of important diseases in relation to the present invention is discussed.

Crohn's Disease

The chronic inflammatory disease Morbus Crohn (Mb. Crohn, Crohn's Disease) is a relatively frequently occurring disease, the prevalence being 55 per 100000 individuals. The incidence has during the last 20 years been increasing by 8-9 new cases per 100000 individuals per year. Diagnosis and treatment of Crohn's disease are therefore a major task for specialised medical gastroenterologic hospitals.

In the past, the treatment of Crohn's disease has been based on an inhibition or modulation of the immune system by means of i.a. azathioprine and cyclosporin. The results obtained by this treatment have been varying, and a way of dividing the disease into subgroups may be needed in order to successfully treat the disease by immune modulation.

Recent research has rendered it possible that Morbus Crohn is a multi-factorial auto-immune disease. It has been suggested that the normal tolerance of the immune system against the microbial flora in the intestines are broken (ref. 6). The chronic immune reactivity against the bacterial flora seems to be mediated by T-lymphocytes producing INFγ and TNFα. The constant presence of these cytokines in increased amounts contributes to the destruction of tissue (an auto-immune reaction) which take place in the inflamed intestine. The treatment of Crohn's disease has accordingly during phase 1 and 2 clinical studies been focused on modulation of the T-cell-mediated immune response by use of IL-10, CD4 antibodies and antibodies against TNFα (refs. 7, 8, 9).

As mentioned above, Crohn's Disease is believed to be a multifactorial disease associated with pro-inflammatory IFNγ and TNFα producing T-cells in the intestinal mucosa. Basically the balance between the pro-inflammatory T-cells and regulatory T-cells is dysregulated resulting in increased production of the pro-inflammatory cytokines. The fundamentals for T-cell vaccination is based on these observations. The pro-inflammatory immune response is activated by different disease relevant antigens. Nevertheless, the activation level in vivo/in situ is not sufficient to activate the regulatory immune response. To stimulate the in vivo regulatory immune response, activated pro-inflammatory T-cells are selected, cultured, activated and attenuated and administered to the patient.

The culture system of the invention selects for the T-cell line with the shortest PD time as shown in example 2. In this case the pro-inflammatory cytokine producing CD4+ T-cell line expands from the gut biopsy on behalf of the regulatory T-cells. In order to avoid the propagation of IL-10 producing regulatory T-cells, which suppress the growth of pro-inflammatory T-cells, selection procedures, as described above can be used. Cyclosporine suppresses the production of IFNγ and TNFα of the in vivo antigen stimulated pro-inflammatory culture (ref. 6). In cultures where cyclosporine is used as a supplement to at least two cytokines, the development of regulatory T-cells is suppressed. Regulatory T-cells are dependent on the presence of IL-10 or/and TGFβ, and in order to establish pro-inflammatory T-cells from intestinal biopsy specimen the selection of pro-inflammatory T-cells may be facilitated by the addition of IL-10 antibody to early cultures. Of course combination of antibody to IL-10 and cyclosporine may also be used.

If the established culture is not sufficiently growing, it can be stimulated with autologous relevant antigen, either intestinal sonicated bacterial material presented by antigen presenting cells (dendritic cells developed from peripheral blood), or by auto-presentation of super-antigen in accordance with the Examples below, or presented with pulsed APCs. To avoid activation induced apoptosis, αCD95 or Z-VAD could be used concomitantly in the culture medium.

The development of dendritic cells is dependent on the presence of GM-CSF and IL-4 (ref. 7). When a sufficient amount of dendritic cells are available (10⁷) 10⁶ γ-irradiated dendritic cells incubated with sonicated bacterial material or super-antigen are mixed with the desired culture in a 1:1 relationship. After 24 hours, positive selection may be performed by usage of either CD69-Ab, CD25-Ab, FAB210 (transmembrane TNFα antibody) or Infliximab (chimeric TNFα antibody with high avidity for transmembrane TNFα).

In patients with severe disease, the activity level of the inflammatory T-cell is pronounced and if the development of IL-10 producing T-cells is avoided, cultures relevant for T-cell vaccination emerges. In cases where the in vivo antigen activation elicits a regulatory T-cell response, selection of pro-inflammatory T-cells by antibodies against the activation markers CD69, CD25 or transmembrane TNFα is an option in the early phase of the culture. Usually regulatory cells do not establish until two to three weeks after the establishment of the culture and the time related dynamics in the culture can be used in the selection process. Magnetic beads coupled to the relevant activation marker antibody may for instance be used.

The expression of surface activation markers and proliferation can also be non-specific augmented by CD3-Ab, CD2-Ab, CD28-Ab. Positive selection after stimulation is performed as described above.

In some cultures the growth of CD4+ cells could be inhibited by CD8+ cells. The CD8+ cells can be removed by negative selection.

When 10⁹-10¹⁰ cells with the relevant phenotype (CD4+, CD45RO+, CD25+, (Act-1)+, CD69+, Transmembrane-TNF+) and function (IFNγ and TNFα production) are available, the cells may advantageously be activated and attenuated by γ-irradiation prior to administration to the patient, for example in the form of an injection subcutaneously in the forearm.

Selection of regulatory T-cells for adjuvant therapy in patients with Crohn's Disease can be achieved by allostimulation with the allogenic T-cell line Se-Ax (cf. the Examples). It is assumed that the pro-inflammatory T-cell recognises the allogenic Se-Ax (an IL-10 producing T-cell line from a patient with Sezary's syndrome). Hereby a pro-inflammatory response inducing secretion of type 1 cytokines stimulate the development of regulatory T-cells (because Se-Ax also produces IL-10 needed to generate CD4+ regulatory lymphocytes). Regulatory T-cells can also be induced by the addition of IL-10 to the culture media also in combination with TGFβ (ref. 10). The autologous regulatory IL-10 producing T-lymphocytes may be used as intravenous adjuvant immunological therapy in patients with active Crohn's Disease.

Different patients with Crohn's Disease may share common peptides in the variable region of the β-chain of the T-lymphocyte receptor site essential for the development of the type ideotype response. In this case peptide libraries from the T-cell receptor V_{β}-chain could be used as a vaccine in Crohn's Disease.

Asthma

Asthma is related to type 2 cytokine producing (IL-4, IL-5, IL-3 and GM-CSF) T-lymphocytes in the bronchial epithelium. The cytokines mobilise and activate eosinofils for subsequent mucosal tissue injury. The same relationship is related to atopic dermatitis. In the bronchial epithelium in patients with asthma, stimulation with house dust mite (HDM) is associated with a type 2 cytokine response with production of IL-4, IL-5 and IL-10. In normal individuals, stimulation of respiratory epithelial T-lymphocytes with HDM elicits a type 1 cytokine response predominated by the production of IFNγ.

Patients with asthma could also be subjects for T-cell vaccination with attenuated type 2 cytokine producing CD4+ T-cells in order to obtain IL-10 producing cells or a type 1 cytokine response reducing disease activity.

The relevant T-lymphocytes could be obtained by either bronchial biopsies or bronchioalveolar lavage and cultured in a medium supplemented with at least two cytokines, and GM-CSF. It would be relevant to use GM-CSF because dendritic cells are very abundant in the respiratory epithelium.

Dendritic cells could also be cultured according to the methods mentioned above, e.g. from peripheral blood cells.

In asthma, it may be relevant to stimulate blood mononuclear cells with known antigens. It has previously been demonstrated, that in patients with severe asthma, CD4 and CD8 enriched peripheral blood expresses spontaneously increased amounts of mRNA for the type 2 cytokines localised to CD4 but not CD8 cells (ref. 11). These CD4+ cells could be stimulated with antigen presented by dendritic cells, developed as mentioned previously, in a medium supplemented with high levels of IL-2 and IL-4 as described by Kaltoft 1998 (ref. 12).

The T-lymphocytes obtained by culture should be described concerning function, avidity (known antigen), and phenotype. Selection procedures as described previously can be used. Cyclosporine has been used in the treatment of asthma. In these patients a down-regulation of the IL-5 response is important, probably because of inhibited IL-5 gene transcription by cyclosporine (and FKS06), but also because of a general down-regulation of calcium dependent transcription of cytokine mRNA (ref. 13). In order to eliminate a type 1 T-cell overgrowth in the established cultures, cyclosporine may be added to the cultures.

In many cases of asthma, the antigen is known (HDM or T-cell reactive peptides in asthma ICP1 and ICP2 epitopes known in cat allergy (synthesised from the cat allergen Fel d1)). In order to stimulate growth, HDM, ICP1, ICP2 or other relevant antigens can be used presented by dendritic cells. In asthma, co-stimulation of the T-cells with dendritic cells via CD28 could be combined with CTLA4-Ig fusion protein because when dendritic cells ligate with CTLA 4 on T-cells, it has been associated with apoptosis.

Multiple Sclerosis

The disease is associated with auto-immune CD4+ T-cells reactive against the myelin, inducing secretion of inflammatory type 1 cytokines in the diseased neural tissue. In multiple sclerosis, T-cell vaccination has been investigated, but so far, it has not been possible to obtain sufficient amounts of activated T-cells with the desired phenotype and cytokine profile. In previous vaccination attempts, the disease associate phenotype, specificity and function of the T-lymphocytes have not been secured (refs. 14, 15).

The culture and selection procedure which could be relevant in these patients are similar to the methods described for Crohn's Disease.

Relevant in vivo disease associated, in vivo antigen activated T-cells could be obtained by spinal puncture. This material could after centrifugation, be propagated in a medium containing at least two cytokines. If growth is not sufficient the T-cells could be activated with myelin as described above.

If sufficient amounts of T-cells cannot be obtained from spinal puncture, peripheral blood mononuclear cells could be separated and isolated by a Ficoll-Isopaque gradient and stimulated by APC presenting myelin.

Continuous CD4+ T-cells with reactivity against myelin and a type 1 cytokine production will after activation and attenuation be ready for T-cell vaccination.

Cancer

For the treatment of cancer, the present invention is believed to be of special interest. Most cancers are associated with tumour infiltrating lymphocytes (TIL), and these TIL's are known to have killer cell activity against the tumour cells. Examples of cancers where this phenomenon are well documented include melanoma, colorectal cancer, renal cell carcinoma, breast cancer and sarcoma.

Although TIL's have anti-tumour activity, the main problem for efficient treatment of cancer with TIL's have so far been that it has not been possible to grow TIL's in sufficient quantities.

TIL's have so far been cultured to approximately 10¹¹ cells (ref. 16) corresponding to approximately 100-300 grams of cells and this quantity has in most cases not been sufficient to combat large tumour masses (in the order of some kg) also partly because not all the cultured TIL's after long term culture do not have the desired specificity against the tumour cells (ref. 16). The present invention overcomes these limitations. So far it has been shown that two continuous CD8+ T-cell lines with specificity and killer cell activity against autologous tumour cells have been established from patients with mycosis fungoides and Sezary's syndrome, respectively.

Examples of cancerous diseases which could be treated with the T-cell lines or T-cells prepared according to the present invention include malignant melanoma, renal carcinoma, breast cancer, lung cancer, cancer of the uterus, prostatic cancer, cutaneous lymphoma and hepatic carcinoma.

As most tumour associated antigens are relatively few (because most tumour associated antigens are self-antigens), the present invention as outlined herein may be used not only to treat the patient from which the lymphocytes derived, but also offers the possibility of treating different but HLA matched patients with these established continuous T-cell lines.

For example in the case of metastatic malignant melanoma, HLA-typing may be performed on peripheral blood cells. If the result of this typing shows that the patient for example expresses HLA-A2 (HLA 0201), the immunogenic melanoma associated peptides restricted by this HLA allele are known to derive from at least the following proteins: Tyrosinase, Melan-A/Mart-1 and gp100. The amino acid sequence of the HLA-A2 binding melanoma associated peptides is for tyrosinase MLLAVLYCL, for Melan-A/Mart-1 AAGIGILTV, and for gp100 KTWGQYWQV. Peptide-MHC tetramers from these melanoma associated peptides can then be used to determine whether the patient has circulating CD8+ T-lymphocytes with specificity to the peptides. Such CD8+ positive cells are also expected to be present in a larger fraction of the outgrowing biopsy derived T-lymphocytes, and the peptide-MHC tetramer technique can thus be used to enumerate and select for melanoma antigen specific T-cells with different avidity among outgrowing biopsy derived T-lymphocytes. Furthermore, immunochemistry of tumour biopsy material can confirm and supplement the data obtained by the peptide-MHC tetramer technique.

The outgrowing T-lymphocytes are in general of oligoclonal origin and consist of both CD4+ and CD8+ T-lymphocytes. Contained within the latter population are the presumed auto-immune effector cells (killer cells), while contained within the former population are CD4+ cells mediating help in generating CD8+ effector cells.

Peripheral blood derived dendritic cells can be pulsed with melanoma associated peptides and used to expand melanoma associated peptide-MHC tetramere selected CD8+ cells in the medium supplemented with IL-2, IL-4, GM-CSF and Z-VAD or functionally similar combinations of growth and selection factors in the presence of γ-irradiated outgrowing T-cells as feeder and helper cells. The desired CD8+ tumour specific lymphocytes may then be expanded according to the procedures aiming at expanding T-lymphocytes in an unlimited number. The specificity and function of the T-lymphocyte cell lines can be confirmed by killing and cytokine production of HLA-matched tumour cells presenting the melanoma associated peptides in question.

If the melanoma associated peptides are not known, but melanoma cells or melanoma cell lysate are available an alternative approach can be employed. Dendritic cells and outgrowing lymphocytes are mixed for some time in a medium containing IL-2, IL-4, GM-CSF and Z-VAD or functionally similar combinations of growth and selection factors. Later tumour cells or tumour cell lysate are added and following expansion, appropriate selection procedures should select for CD8+ cells with tumour cell reactivity. It should be noted that continuous T-cell lines are often oligoclonal for more than 100 PD, implying that continuous CD8+ tumour specific T-lymphocyte cell lines may react with several melanoma associated antigens, thus minimising the risk of tumour escape.

Selection for melanoma specific CD8+ cells may also be obtained by mixing outgrowing T-lymphocytes with tumour cells in a medium with IL-2, IL-4 and Z-VAD or functionally similar combinations of growth and selection factors, because the tumour cells (target cells) acts as antigen presenting cells by directly presenting tumour associated peptides to CD8+ T-lymphocytes.

When continuous CD8+ T-cell lines are available, these cell lines can be used to treat HLA-matched melanoma patients tumour associated antigens recognised by the continuous CD8+ cell lines (including of cause the patient from which the continuous cell lines derive), in particular patients with metastatic malignant melanoma. In the case described above melanoma patients with HLA-A2, an allele which more than 40% of Caucasian melanoma patients carry. Patients with metastatic malignant melanoma have a very poor prognosis with a median survival time of only 7.5 months. Accordingly it is desirable to have access to treatment options that can work fast like pre-made continuous HLA-matched tumour specific CD8+ cell lines. As the vast majority of HLA-matched melanoma patients express the same tumour associated antigens, it may be possible to establish a T-lymphocyte cell bank that optimally will fit every patient with malignant melanoma regarding tumour cell killing and HLA-match for non-presenting HLA-alleles.

The tumour specific CD8+ T-lymphocytes may be γ-irradiated in order to ensure that the cells cannot divide further and infused to the patient in combination with an established IL-2 therapy protocol. Before administration, e.g. infusion, the T-lymphocytes can be incubated with the caspase inhibitor Z-VAD, in order to reduce AICD, or Z-VAD may be given during the administration. Like other TIL's, the continuous CD8+ cell lines are expected to home to the tumour bed, thereby initiating a massive tumour cell destruction followed by cytokine production located at the tumour sites. Besides killing of the tumour, AICD is expected to lead to a fast elimination of the administered lymphocytes, which in general should be sensitive to Fas-FasL killing in order not gain access to immune privileged sites such as the eyes and the testis. Due to the elimination of lymphocytes, large quantities are obviously needed to combat large tumour masses. As soluble melanoma associated peptide HLA complexes are released during melanoma cell killing, such complexes interfere with the interaction between CD8+ cells and melanoma cells. Thus, it may be necessary to remove such complexes from the blood stream during treatment for instance by an immuno-magnetic separation technique. The presence of soluble melanoma peptide HLA complexes can however serve as a marker for the effectiveness of tumour eradication. Furthermore, when allogenic cytotoxic cells differ from the patient's HLA-type, this may be used to follow the number and fate of the infused lymphocytes.

It is expected that the administered attenuated, for example infused, e.g. γ-irradiated, CD8+ lymphocytes are capable of killing, if not all, then the vast majority of tumour cells. Furthermore the inflammation generated by the administered CD8+ cells (perhaps also with the addition of administered helper CD4+ cells) will activate autologous resident but inactive melanoma specific pre-cytotoxic T-cells to killer cells, in part due to the expansion/maturation of dendritic cells that are activated by the production of GM-CSF and TNFβ during melanoma cell killing.

In combination, the above effect mechanisms are expected to eradicate all tumour cells.

Pharmaceutical Compositions

The present invention also relates to pharmaceutical compositions comprising activated disease associated T-cells prepared according to the methods described herein, or comprising one or more T-cell lines as described herein, optionally comprising one or more pharmaceutically acceptable drugs and/or excipients.

The T-cells to be used in the composition are preferably inflammatory T-cells, regulatory T-cells, or cytotoxic T-cells.

In one embodiment, the composition comprises T-cells or one or more T-cell lines which have been re-activated in the presence of one or more antigens. Such antigens may preferably be disease associated antigen(s), alloantigen(s), or super-antigen(s). Examples of super-antigens are SEA, SEB, SEC, SED, SEE, TSST, Streptococcus pyogenes enterotoxin A, B and C, and Mycoplasma arthritidis antigen. Disease associated antigen(s) can be added in the event the antigen is known. Alternatively, re-activation may be carried out with a tissue sample or another sample expected to comprise the disease associated antigen.

The T-cells are preferably attenuated prior to administration in order to ensure that the cells are not able to divide further. Such attenuation may suitably be accomplished by x-ray or UV radiation or by addition of cell poisons.

The suitable amount of the T-cells of the invention to be administered depends on several factors, i.a. the disease or condition to be treated, alleviated or prevented, and further on the age, weight and state of the subject to be treated. The skilled person art will readily know how to establish the optimum dose.

The administration may be as single doses or as several doses per day. In certain cases, administration only once may be sufficient. In general, several doses should be given such as once for a period of for examples a day for a week or for months, or repeated administration once every week, every second week, etc.

The amount of the T-cells of the invention depends on patient, on the route of administration, and the severity of the disease or condition to be treated. In general, 10⁸-10¹² cells may be suitable for each dose.

The pharmaceutical composition is conveniently administrated parenterally, by injection either subcutaneously, intramuscularly, intravenously or by infusion.

For V_{β} disease specific peptides, injectables may be in the form of liquid suspensions or solutions, solid forms suitable for solubilisation or suspension in liquid prior to injection. The pharmaceutical composition may also be emulsified. Additional modes of administration may in certain cases be suitable such as e.g. oral formulations.

The pharmaceutical composition may also be mixed with suitable excipients such as e.g. water, saline, dextrose, glycerol, ethanol or combinations thereof. In addition, the composition may contain auxiliary substances such as wetting agent, emulsifying agents, colouring substances, preserving agents, or pH buffering agents.

Vaccines

T-cell vaccination seems to be an attractive treatment of various diseases including auto-immune diseases and cancer. However, in practice, T-cell vaccination has not been a realistic option since auto-reactive T-cells as other humane T-lymphocytes are believed to have a limited dividing capacity in vitro.

One problem is that it has not been possible to obtain a sufficient number of cells to perform vaccination. By the present invention, an unlimited number of cells is available, thus, making T-cell vaccination possible.

Accordingly, in another aspect, the present invention relates to vaccines comprising activated disease associated inflammatory T-cells prepared in accordance with the methods described herein, or one or more T-cell lines as described herein.

In one embodiment of the vaccine, the T-cells have been re-activated in the presence of one or more antigens. Representative examples of such antigens are disease associated antigen(s), alloantigen(s), or super-antigen (s). Examples of super-antigens are SEA, SEB, SEC, SED, SEE, Streptococcus pyogenes enterotoxin A and B, and Mycoplasma arthritidis antigen.

In a preferred embodiment of the vaccine, the T-cells have been attenuated. Such attenuation may suitably be accomplished by γ- or UV-radiation, or by addition of cell poisons.

Disease associated antigen(s) can be added in the event the antigen is known. Alternatively, re-activation may be carried out with a tissue sample or another sample expected to comprise the disease associated antigen.

The T-cells are preferably attenuated prior to administration in order to ensure that the cells are not able to divide further. Such attenuation may suitably be accomplished by x-ray or UV radiation or by addition of cell poisons.

The suitable amount of the T-cells of the invention to be administered depends on several factors, i.a. the disease or condition to be treated, alleviated or prevented, and further on the age, weight and state of the subject to be treated. The skilled person art will readily know how to establish the optimum dose.

The administration may be as single doses or as several doses per day. In certain cases, administration only once may be sufficient. In general, several doses should be given such as once for a period of for examples a day for a week or for months, or repeated administration once every week, every second week, etc.

The amount of the T-cells of the invention depends on patient, on the route of administration, and the severity of the disease or condition to be treated. In general, 10⁸-10¹² cells may be suitable for each dose.

The pharmaceutical composition is conveniently administrated parenterally, by injection either subcutaneously, intramuscularly, intravenously or by infusion.

For V_{β} disease specific peptides, injectables may be in the form of liquid suspensions or solutions, solid forms suitable for solubilisation or suspension in liquid prior to injection. The pharmaceutical composition may also be emulsified. Additional modes of administration may in certain cases be suitable such as e.g. oral formulations.

The pharmaceutical composition may also be mixed with suitable excipients such as e.g. water, saline, dextrose, glycerol, ethanol or combinations thereof. In addition, the composition may contain auxiliary substances such as wetting agent, emulsifying agents, colouring substances, preserving agents, or pH buffering agents.

Prior to vaccination with the vaccine of the present invention, or treatment with the pharmaceutical composition of the present invention, the phenotype for T-lymphocyte receptors TCR-α,β and TCR-γ,δ of the cell culture may be determined e.g. by flow cytometry. Likewise, the HLA-DR, CD3, CD4, CD8, CD11, CD18, CD23, CD28, CD45RO, CD54, HML-1 CD11a and clonality characteristics may be determined, providing important information.

Furthermore, the cytokine profile may be determined. The following cytokines may be determined: INFγ, IL-10, TNFα, IL-12, IL-2, IL-4 and TGFβ. Also, extended HLA class I and II as well as status regarding Hepatitis A, B, and C, and HIV, CMV, EBV and HTLV-I should be determined for both the subject and the cell lines. Also, intracellular amount of NFκB and JAK/STAT pathway may be monitored.

Uses of the T-Cells or T-Cell Lines of the Present Invention

Furthermore, the use of the T-cell lines and T-cells as described and claimed herein of a medicament for the treatment of a T-cell associated disease also forms part of the present invention. In particular, the medicament is used for treating, alleviating or preventing diseases of inflammatory, auto-immune or neoplastic origin, or combinations thereof. Examples of such diseases are given above. In particular, the medicament may be for treating, alleviating, or preventing inflammatory bowel disease, Crohn's colitis, sclerosis, type I diabetes, rheumatoid arthritis, psoriasis, atopic dermatitis, malign melanoma, renal carcinoma, breast cancer, cutaneous lymphoma, or the like.

In addition to the content of T-cells, the compositions or vaccines may contain drugs for use in a conventional treatment of the particular disease, or drugs for the treatment or prevention of side effects in connection with the disease or treatment of the disease. Such drugs should readily be known to the practitioner (doctors etc.). Examples are 5-aminosalicylic acid, azathioprin, Prednisone, budesonide.

Diagnosis

In yet another aspect, the present invention relates to a method for the diagnosis of a disease in a mammal, which method comprises
(a) obtaining a tissue sample from a mammal including a human being, the sample comprising activated T-cells, antigen presenting cells, and antigen(s),
(b) culturing said tissue sample or said activated T-cells in the presence of two or more T-cell growth factors and optionally one or more additional compound,
(c) observing the presence of disease associated T-cells, and relating the presence of these T-cells to a disease.

In one embodiment of the diagnostic method, the disease is related to the disease associated T-cells by determining the kind or phenotype of the activated T-cells and/or their state of activation.

In another embodiment of the diagnostic method, the cytokine profile of the T-cells is determined. Thereby, the activated T-cells are determined, and thus, the disease.

Methods for the Treatment, Alleviation or Prevention of Diseases Associated with Activation of T-Cells

In a special aspect, the present invention relates to a method for the treatment, alleviation or prevention of a disease associated with an activation of T-cells in a subject comprising administering to the subject one or more T-cell lines, T-cells, a composition or a vaccine as defined and claimed herein.

Such method comprises
(a) obtaining a tissue sample from a mammal including a human being, the sample comprising disease activated T-cells, or
   obtaining T-cells and antigen-presenting cell from said mammal and mixing said cells with a disease associated antigen or antigens, and
(b) culturing said tissue sample or said mixture of cells and antigen(s) in the presence of at least two factors promoting T-cell growth and optionally one or more additional compound.

Factors that promote T-cell growth are given above and include cytokines that promote T-cell growth. Examples are IL-2, IL-4, IL-7, IL-9, IL-10, IL-15, IL-16, and functionally similar compounds. In particular embodiment, a combination of IL-2 and/or IL-15 and IL-4 and/or IL-7 is used, preferably a combination of IL-2 and IL-4. The concentration of the cytokines may preferably be at least 1 nM, more preferably more than 2.5 nM, and most preferably more than 10 nM.

The method of expanding and selecting the disease associated T-cells are described in greater detail above.

The sample to be cultured may be a tissue sample or another sample as defined above. The sample from which the T-cells are expanded may in one embodiment be a tissue sample collected from the patient to be treated, and in another embodiment a tissue sample collected from a patient different to the patient to treated. Furthermore, the HLA restriction of the T-cells and in the patient to be treated may be determined.

Diseases to be alleviated, prevented or treated are in particular those described above.

Furthermore, the invention relates to a method for the treatment, alleviation or prevention of a disease associated with an activation of T-cells in a subject comprising administering a medicament as identified according to the method identified as being effective in said treatment.

The disease is a disease of inflammatory, auto-immune, allergic, neoplastic or transplantation-related origin, or a combination of such. In accordance herewith, the disease may be an inflammatory bowel disease such as Crohn's colitis or ulcerative colitis, sclerosis, type I diabetes, rheumatoid arthritis, psoriasis, atopic dermatitis, asthma, malignant melanoma, renal carcinoma, lung cancer, cancer of the uterus, prostate cancer, hepatic carcinoma, breast cancer, cutaneous lymphoma, rejection-related disease or Graft-versus-host-related disease.

Accordingly, candidate factors are tested in a method as described herein in place of IL-2 or IL-4 or a functionally similar compound or in addition to the combination of IL-2 and IL-4 or said functionally similar compound(s) and the effect compared to the effect obtained by using a combination of IL-2 and IL-4.

Methods of Testing the Effect of a Medicament

The present invention also relates to a method of testing the effect of a medicament against a T-cell associated disease, which method comprises
(a) providing a T-cell line as defined above,
(b) applying the medicament to be tested to the T-cell line, and
(c) observing the effect of the medicament on the T-cell line.

In one embodiment of this method, the cytokine profile of the T-cell line with and without the addition of the medicament is compared. Furthermore, the phenotype, proliferation and/or apoptosis of the T-cell line with and without the addition of the medicament may be compared In particular, the intracellular amount of NFκB and/or JAK/STAT pathway may be monitored.

In this method, the medicament to be tested is preferably selected from compound libraries such as small molecule libraries or peptide libraries or antibodies against T-cell components. In particular, the medicament may be selected from peptide fragments from T-cell receptors.

Model Systems

Thus, in a further aspect, the present invention relates to a model system for testing the effect of a medicament against a T-cell associated disease, which model system comprises at least one T-cell line as defined above.

Methods of Detecting T-Cell Growth Factors

The invention also relates to a method of detecting T-cell growth factors for use in the method of expanding and selecting disease associated T-cells as defined above, in which method candidate factors are used in place of IL-2 or IL-4 or in addition to the combination of IL-2 and IL-4, and in which the effect compared to the effect obtained by using a combination of IL-2 and IL-4.

Methods of Monitoring Responses

The present invention also includes a method of monitoring the response to a treatment of a disease of inflammatory, auto-immune or neoplastic origin, or combinations thereof, said method comprising comparing the phenotype, proliferation, apoptosis, and/or cytokine profile of activated T-cells in tissue sample taken from the patient to be treated before the start of the treatment and during the treatment and/or after the treatment has ended. Accordingly, this method may be used to identify patients which do not responding to a certain treatment.

Methods of Identifying Disease Associated Antigens

A part of the present invention is also a method of identifying disease associated antigens, comprising screening peptide libraries or antigen samples for their re-activation properties in a T-cell line as defined and claimed herein.

The present invention is further illustrated by the following non-limiting examples.

### EXAMPLES

### Example 1

Derivation of Finite and Continuous Peripheral Blood T-Cell Lines

Peripheral blood mononuclear cells (PBMC) from 3 healthy donors were isolated by standard Ficoll-Isopaque gradient centrifugation. The PBMC were resuspended at 5×10⁵ cells/ml in 90% RPMI 1640, 10% human AB serum, 1000 u/ml IL-2 and 500 u/ml IL-4 with antibiotics as described (ref. 12). To access whether longevity of cultured PBMC is dependent on in vitro activation, PBMC were cultured in the above medium alone or with additional alloactivation. 5×10⁶ PBMC were stimulated with the heavily γ-irradiated (60 Gy) Psor-2 cell line at a 5:1 ratio. The Psor-2 cell line is a continuous T-cell line established from a skin biopsy specimen of a patient with psoriasis vulgaris by culturing the skin specimen in the medium mentioned above (ref. 0).

Estimation of CD28 expression as a function of cell population doublings. Monoclonal antibodies against CD3, CD4, CD8, CD28, and CD56 were purchased from PharMingen. An α/β T-cell receptor subfamily antibody against V_{β}18 was obtained from Immunotech. An indirect immunofluorescence technique was applied to label the cells as previously described (ref. 12). Allostimulated continuously growing peripheral blood T-cell lines were cryopreserved for each 10 PD. Cells cryopreserved at different PD were then thawed, cultured for 4 days and analysed for CD28 expression by flow cytometry. CD4 and CD8 expression served as positive and negative controls, respectively. For each antibody, 2×10⁴ cells were analysed (FACS Calibur, Becton Dickinson). Fluorescence microscopy was also applied to evaluate the stainings.

A clonal CD4+, V_{β}18+ T-cell line My-La, 46,XY,i(18q) (refs. 17, 18) cultured with 1000 u/ml IL-2 and 500 u/ml IL-4 was also analysed for CD28 expression at different PD.

Other methods. Cells were found to be free of mycoplasma by the Hoechst staining test. Telomerase activity of 10³ cells was determined by the TRAPeze Telomerase Detection Kit as described by the manufacturer (Oncor).

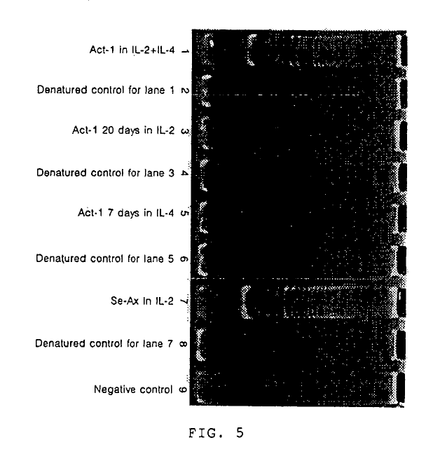

Growth of peripheral blood cells with and without allostimulation. PBMC from the 3 healthy donors proliferated between 1 to 3 PD when cultured in the cytokine supplemented medium alone (FIG. 4) in agreement with previously published data showing that peripheral blood cells proliferate only transiently when stimulated with a combination of IL-2 and IL-4 (refs. 4, 12). However, when PBMC were allostimulated once with the Psor-2 cell line in the presence of a high concentration of IL-2 and IL-4, T-cells as well as non-T-cells (preferentially CD3−, CD56+) proliferated vigorously during the first 4 to 6 weeks.

After approximately 50 PD only CD4+ T-cell grew in the cytokine based medium. All three CD4+ allostimulated T-cell lines have proliferated beyond 150 PD with a PD-time of 30 to 36 hours (FIG. 4). This corresponds to an increase in cell numbers of 2¹⁵⁰ 10⁴⁵-fold. As allostimulated peripheral blood T-lymphocytes have been estimated to have a limited in vitro life-span of 23±7 PD (ref. 19) the allostimulated CD4+ cell lines reported here can be considered continuous, effectively having an unlimited replication capacity.

So far, the three continuous peripheral blood derived CD4+ cell lines show no sign of growth exhaustion and at PD 150 still retain alloreactivity (results not shown).

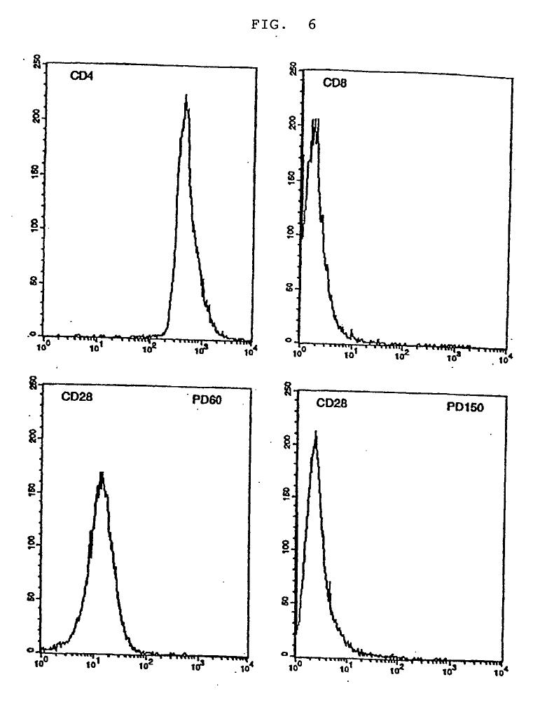

Cytokine dependent continuous T-cell lines have cytokine dependent telomerase activity. Continuous cell lines are expected to have telomerase activity. When cultured in the presence of both IL-2 and IL-4 in vitro activated peripheral blood CD4+ T-cells show high telomerase activity (FIG. 5) comparable to that of a leukemic cell line Se-Ax (ref. 20), established form a patient with Sezary's syndrome. Withdrawal of either IL-2 or IL-4 results in growth arrest. After withdrawal of IL-4, a 100 PD cell culture cease proliferating after 14 to 21 days. Withdrawal of IL-2 results in cell growth arrest between 6 to 9 days. As shown in FIG. 5 telomerase activity in IL-2 or IL-4 starved cells is severely reduced. The results indicate that simultaneous presence of IL-2 and IL-4 regulates both growth and telomerase activity in these T-cell lines.

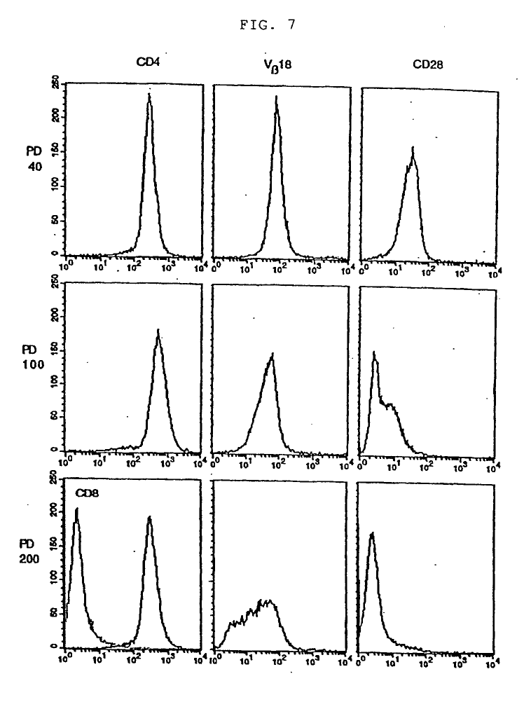

CD28 expression correlates inversely with cell population doublings. Allostimulated PBMC cultured in the cytokine supplemented medium became pure CD4+ cell lines after approximately 50 to 60 PD. CD28 expression of one such CD4+ cell line, Act-1, at PD 60 and PD 150 is presented in FIG. 6. CD28 expression is clearly detectable at PD 60 but absent at PD 150. A gradual decline in expression of CD28 between PD 60 and PD 150 could be observed.

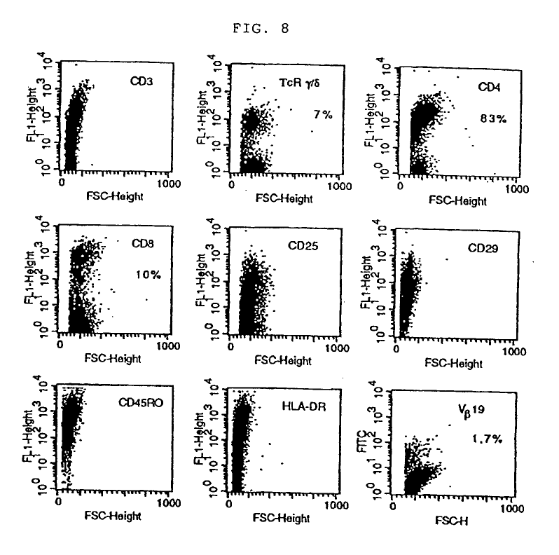

To investigate whether the culture system preferentially expands pre-existing CD28 negative CD4+ cells or whether CD28 could serve as a mitotic clock in individual T-cells a clonal CD4+, V_{β}18+ T-cell line established from an inflammatory skin biopsy specimen (refs. 17, 18) was investigated for CD28 expression. As shown in FIG. 7, CD28 expression of this T-cell clone (My-La, 46,XY,i(18q)) decreases gradually with cell population doublings being present at PD 40 and completely absent at PD 200. However, CD4-+expression is compatible at PD 40 and PD 200. These findings are in agreement with data obtained from finite CD4+ T-cell lines (ref. 5) showing down-regulation of CD28, but not complete loss of CD28 expression with increasing PD. The results presented here show that CD28 expression correlates inversely with cell population doublings and indicates that CD28 expression can serve as a mitotic clock at the clonal level.

The results show that alloactivation with the continuous psoriatic T-cell line Psor-2 can efficiently prime allo-generic CD4+ peripheral blood T-cells to cytokine dependent continuous growth. These cytokine-driven peripheral blood derived CD4+ T-cell lines show IL-2 and IL-4 dependent telomerase activity, and they gradually loose CD28 expression with increasing cell population doublings.

Conclusion. Contrary to other normal human somatic cells T-lymphocytes can in vitro like in vivo be activated to continuous cytokine driven growth. The results presented here raises the possibility of generating an unlimited number of T-cells with predefined specificity. Such immortal T-cell lines may be useful for several applications, for instance for standardisation of T-cell mediated biological assays and for generating sufficient numbers of auto-immune T-cells for human T-cell vaccination.

### Example 2

Super-Antigen Directly Augment the Cytokine Production of Two Novel Continuous Gut-Derived T-Cell Lines from Patients with Crohn's Disease

IFNγ producing CD4+ T-lymphocytes have been implicated with progression of Crohn's disease whereas IL-10 producing CD4+ T-lymphocytes are thought to down-regulate disease activity.

In the following, it is investigated whether a newly devised cell culture protocol could select for continuous clonal CD4+ T-cell lines producing either IFNγ or IL-10.

Biopsy specimens. At least eight colonic biopsies were obtained from affected mucosa of two patients. The biopsies were examined for histopathological changes and a diagnosis of Crohn's disease was established according to clinical, radiological and histopathological data.

In each patient, two additional biopsies were taken for in vitro culture of T-cells. The Gut_{I}-1 T-cell clone was established from a patient undergoing cyclosporine treatment with a CDAI index of 296 whereas the patient from whom Gut_{R}-2 derived had a CDAI index of 155. The study was approved by the local ethic committee.

Cell culture. The two biopsies were washed twice in sterile PBS and once in the growth medium. The growth medium consisted of 90% RPMI 1640 10% human AB serum. 100 U/ml penicillin G 100 μg/ml streptomycin (basal medium, BM) supplemented with 2000 u/ml IL-2 and 500 u/ml IL-4 (complete medium). The T-lymphocytes were initially expanded in 5 ml complete medium and when cell density reached 1.5×10⁴/ml, the culture was split at a 1:2 ratio.

T-cells of the primary cultures from which Gut_{R}-2 derived were allostimulated with the heavily γ-irradiated (60Gy) leukemic cell line Se-Ax at a 5:1 ratio. The continuous Se-Ax cell line was established from a patient with Sezary's syndrome (ref. 20).

Phenotyping

Phenotyping. Monoclonal antibodies against CD3 (OKT3), CD4 (OKT4), CD8 (OKT8) and CD25 were obtained from hybridomas from American Type Culture Collection (ATCC) Monoclonal antibodies against CD45RO and HLA-DR were purchased from PharMingen. Monoclonal antibodies against TCR-1 (TCRγ/δ), TCR-2 (TCRα/β) and α/β T-cell receptor subfamily antibodies against V_{β} 1, V_{β} 2, V_{β} 3; V_{β} 5.1, V_{β} 5.2, V_{β} 5.3, V_{β} 7, V_{β} 8, V_{β} 9, V_{β} 11, V_{β} 12, V_{β} 13.1, V_{β} 13.6, V_{β} 14, V_{β} 16, V_{β} 17, V_{β} 18, V_{β} 19, V_{β} 20, V_{β} 21.3, V_{β} 22 and V_{β} 23 were obtained through Coulter. An indirect immunofluorescence technique was applied to label the cells as previously described (ref. 12). 2×10⁴ events were analysed by flow cytometry (FACS Calibur, Becton Dickinson) and debris and aggregates were excluded by gating. Fluorescence microscopy was also applied to evaluate the stainings.

Stimulation of cells. Cells cultured in complete medium were washed twice with RPMI 1640 in order to eliminate residual cytokines. They were then re-suspended in basal medium with IL-2 or complete medium at 10⁴/ml. Cells were then stimulated either with 10 μg/ml monoclonal antibodies against CD3 or with staphylococcus enterotoxins A, B, D and E at a concentrations of 1 μg/ml (obtained from Toxin Technology Madison, Wis.).

Cytokine determination. Supernatant of stimulated cells and cells cultured in basal medium with IL-2 or complete medium was harvested after 24 or 48 hours. Cytokine matched antibody pairs for determination of IFNγ IL-4, IL-10 and tumour necrosis factor (TNFα) were obtained from Endogen. The detecting antibodies were all biotinylated. A time resolved fluorometric assay applying Europium labelled streptavidin and a Delphia 1234 fluorometer was used to determine the cytokine contents as described by the manufacturer (Wallac). As the cell culture medium contained human serum cytokine, concentrations below 100 pg/ml were not considered to be associated with cytokine producing T-cells. The data were analysed by a computer programme (Biosoft, Assay Zap).

Other methods. Cells were found to be free of mycoplasma by the Hoechst staining test. Karyotyping with Q banding followed standard procedures. The karyotypes were established according to the International System for Human Cytogenetic Nomenclature (ISCN) (1985).

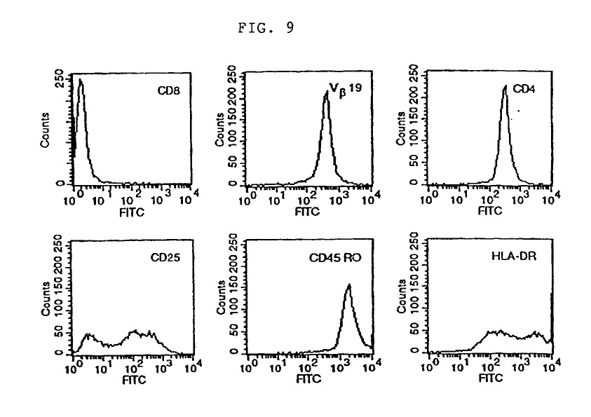

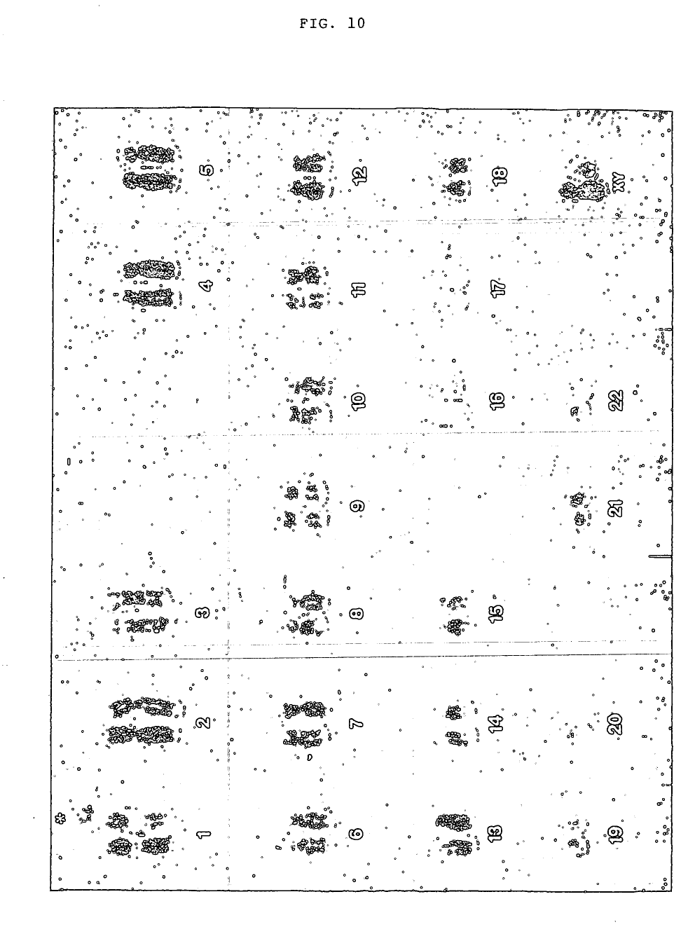

Establishment, phenotype and constitutive cytokine production of Gut_{R}-2. When placed in the complete medium, lymphocytes migrated from the biopsy specimens and proliferation was evident within a week. After approximately two weeks the cell culture had expanded to more than 50×10⁴ cells. The phenotype of this culture is shown in FIG. 8. Both TCR-1 and TCR-2 as well as CD4+ and CD8+ T-cells that are present in situ (ref. 21) are expanded in the cell culture medium. The TCR-2 population was oligo- or polyclonal as evidenced by their reaction with several V_{β} subfamily antibodies. A positive staining with a V_{β} subfamily antibody ranged from 0.2% to 8%. The activation marker CD25 is only partially expressed in the growing T-cell culture (FIG. 8) and another activation marker HLA-DR differs widely in expression among individual T-cells. At this stage, the culture was split in two, half of the cells were cultured with additional allostimulation, the other half was cultured in the complete medium alone. Cells kept in complete medium without allostimulation developed into a finite cell culture dominated by CD8+ T-cells. The allostimulated culture initially also increased the percentage of CD8+ cells. However, after a period with no apparent T-cell number increase, CD4+ T-cells started to proliferate continuously. This CD4+ T-cell line Gut_{R}-2 has proliferated beyond 250 cell population doublings (PD) with a PD time of approximately 36 hours. As allostimulated T-cell lines have been reported to have a finite life-span of 23±7 PDs, Gut_{R}-2 can be considered immortal effectively having an unlimited replicative capacity. At PD˜150 Gut_{R}-2 became independent of IL-4 for continued growth. The phenotype of the continuous Gut_{R}-2 cell line is presented in FIG. 9. Among the V_{β} subfamily antibodies tested Gut_{R}-2 only expresses the V_{β}19 subfamily of the TCR-2 complex indicating that Gut_{R}-2 is a clone. This assumption was confirmed by karyotyping as Gut_{R}-2 after approximately 125 PD developed a clonal chromosome aberration observed in all metaphases (FIG. 10). Thus, also by cytogenetic criteria the V_{β}19+ Gut-2 cell line is a clonal T-cell line. Comparison of FIG. 8 and FIG. 9 shows that clonal Gut_{R}-2 CD4+ T-cell line develops from V_{β}19+ T-cells that comprise less than 2% of the T-cells in the primary culture. As shown in Table 1 the V_{β}19+ clonal Gut_{R}-2 T-cell line constitutively produces IL-10 in basal medium with IL-2 (and also in complete medium), but without additional stimulation. IL-10 concentrations have been measured over a time period of four months corresponding to an increase in cell numbers of approximately 2 [symbol] ˜10²⁴-fold.

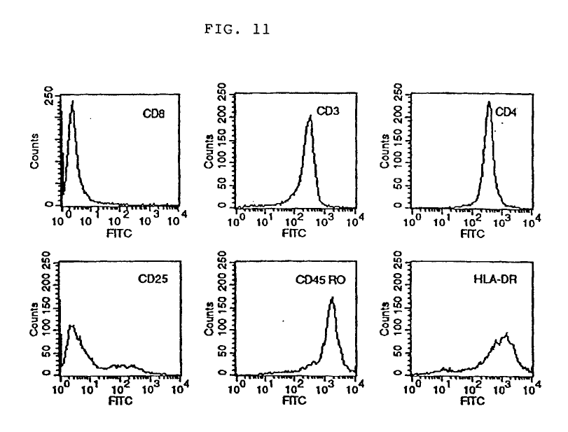

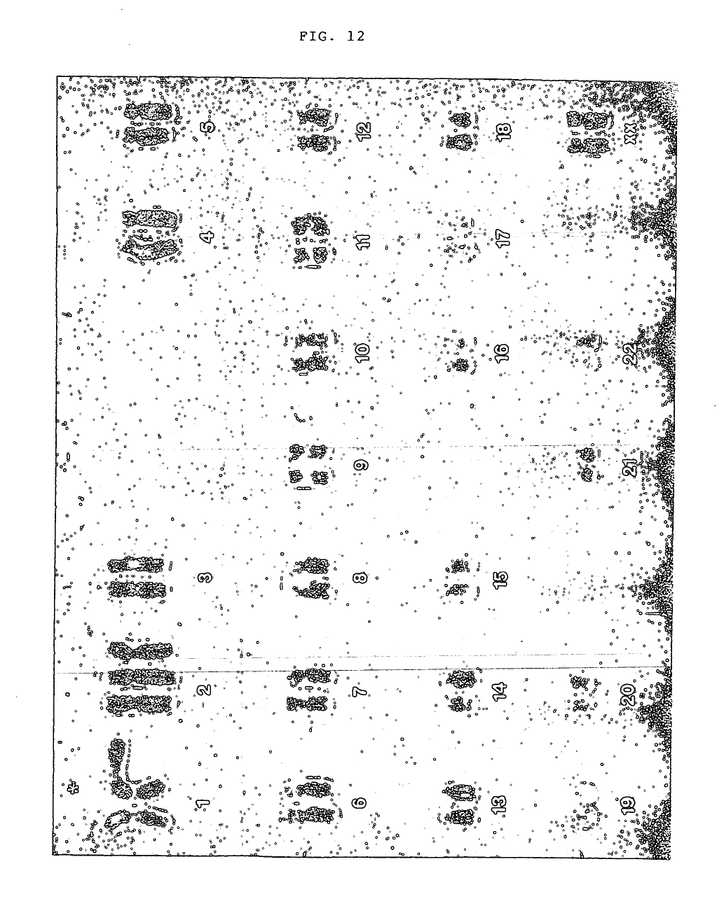

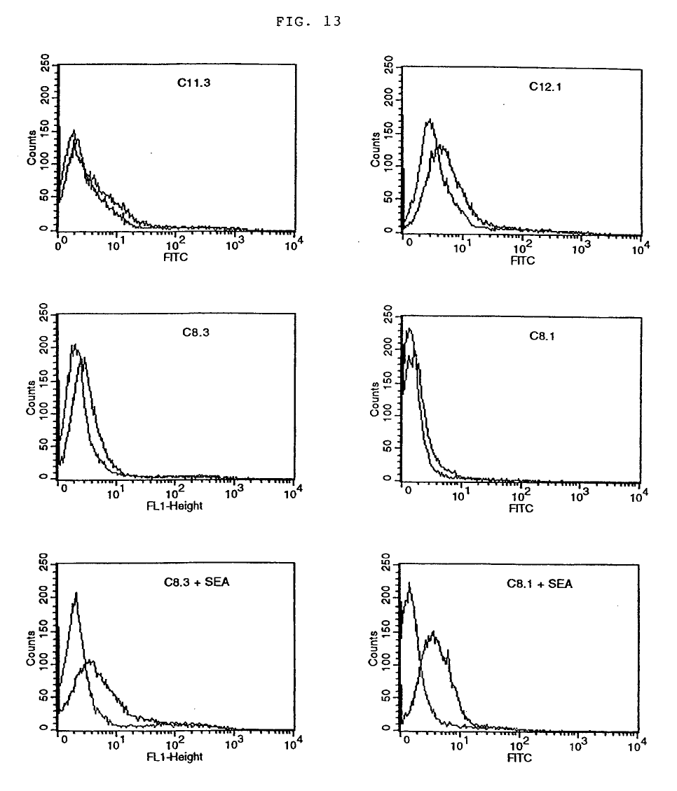

Establishment, phenotype and karyotype of Gut_{I}-1. Within ten days lymphocytes from the gut biopsy specimens from which Gut_{I}-1 derived had expanded to more than 50×10⁴ cells with a phenotype distribution similar to that shown in FIG. 8. Upon culture in the cytokine based medium, but without antigen and accessory cells added, CD4+ T-cells continued to expand, and within 20 PD a pure CD4+ T-cell line evolved that have proliferated beyond 300 PD with a PD time of approximately 30 hours. Thus, this cell line Gut_{I}-1 can be considered continuous. The phenotype of Gut_{I}-1 at PD 150 is presented in FIG. 11 and, as shown, it has markers compatible with mature memory CD4+ T-cells. At PD˜100, Gut_{I}-1 developed a clonal chromosome aberration as shown in FIG. 12 and like Gut_{R}-2, Gut_{I}-1 is also a continuous clonal CD4+ cell line. By phenotyping non of the available subfamily V_{β}, specific antibodies reacted with Gut_{I}-1. Unlike Gut_{R}-2 constitutive cytokine production was not detectable in Gut_{I}-1 cells.

Super-antigens directly induce cytokine production in Gut_{I}-1 cells and augment cytokine production in Gut_{R}-2 cells. As Gut_{I}-1 (and Gut_{R}-2) expresses major histocompatibility complex class II (MHC class II) antigens that are high affinity receptors for several super-antigens, it was investigated whether these cell lines could somehow auto-present super-antigens. Four arbitrarily chosen super-antigens SEA, SEB, SED and SEE were tested for their ability to induce cytokine production in Gut_{I}-1 cells (Table 2). As shown, soluble antibody against CD3 (OKT3) in the presence of IL-2 and IL-4 could not induce detectable cytokine production whereas SEA, SED and SEE induced IFNγ production.

Similarly, the four super-antigens were tested for their ability to alter the cytokine production of Gut_{R}-2 cells. As shown in Table 3, SEB induced high levels of IFN-γ production and also significantly augmented IL-10 production in Gut_{R}-2 cells. As SEB activation is selectively induced in T-cells bearing V_{β}3,12,14,15,19 and 20 (ref. 22) the results presented in Table 3 indicate that Gut_{R}-2 auto-present SEB as classical antigen presenting cells.

Discussion. It has been suggested that the normal tolerance to commensal intestinal bacterial antigens or super-antigens is broken in Crohn's disease. Activated CD4+ T-lymphocytes secreting IFNγ, thereby activating monocytes/macrophages to enhanced TFNα production has been implicated in maintenance of Crohn's disease (ref. 23).

Gut_{I}-1 is an inflammatory CD4+ T-cell clone established from a gut biopsy specimen without addition of mitogen, antigen and accessory cells. It is thus very likely that Gut_{I}-1 was activated in vivo to cytokine driven growth in vitro. This assumption is compatible with the notion that inflammatory T-cells are highly activated in Crohn's disease. It should be noted that the cell culture system selects for the fastest growing T-cell clone implicating that several T-cell clones with properties like Gut_{I}-1 exist in the inflamed gut mucosa. The V_{β} subfamily specificity of Gut_{I}-1 could not be determined by phenotyping excluding the possibility of pre-selecting a super-antigen that could optimally induce IFNγ production. However, Gut_{I}-1 responded by direct addition of SEA, SED and SEE with IFNγ production indicating that Gut_{I}-1 can auto-present super-antigens. Thus, IFNγ production by Gut_{I}-1 cells does not necessarily require a specific antigen presented by antigen presenting cells.

If this property is also reflected in vivo, no specific microbial agent may be essential for the inflammatory response. Furthermore, inflammatory T-cells bypassing the classical antigen presentation could aggravate a chronic inflammation.

Gut_{R}-2 is a CD4+ V_{β}19+ cell clone established by allostimulation of outgrowing gut T-lymphocytes. During a period of nine months without allostimulation (150 PD) the clonal Gut_{R}-2 cell line has constitutively produced IL-10.

As Gut_{R}-2 expresses both high affinity receptors for SEB (MHC class II), and a SEB responsive V_{β} chain (ref. 12) direct addition of SEB to Gut_{R}-2 results in a dramatic IL-10 and IFNγ production. The cytokine production of activated Gut_{R}-2 cells thus resembles a recently described regulatory CD4+ T-cell subset (ref. 24).

It is intriguing to speculate that regulatory T-cells like Gut_{R}-2 with constitutive IL-10 production independent of direct antigen activation may contribute to normal gut tolerance. Gut_{R}-2 shows as mentioned above some properties with a newly described regulatory IL-10 producing CD4+ T-lymphocyte population (ref. 24). However, Gut_{R}-2 differs from this sub-population by constitutive non antigen mediated IL-10 production and by its continuous growth.

An advantage of the cell culture system described here for gut T-cell clones is that their continuous growth gives rise to an unlimited number of T-cells. Such immortal T-cell clones may be useful for testing biological response modifiers, and inflammatory T-cell clones like Gut_{I}-1 could provide the basis for a T-cell vaccination of patients with Crohn's disease.

TABLE 1
Average cytokine production (pg/ml/10 T-cells)
of five different experiments between PD 150 to PD 225 of
continuous growing GUTₖ-2 cells.

| IL-4 | IFNγ | IL-10 | TNFα |
|---|---|---|---|
| <100 | 258 (147-369) | 2460 (1887-3033) | <100 |

Cells in basal medium with IL-2. 95% confidence intervals in parenthesis.

TABLE 2
Cytokine production (pg/ml/10 T-cells) in GUT₁-1
after stimulation with superantigens (at PD 120).

| GUT₁-1 | TNFα | INFγ | IL-10 |
|---|---|---|---|
| Complete | <100 | <100 | <100 |
| medium | | | |
| +antibody | <100 | <100 | <100 |
| against CD3 | | | |
| +SEA | <100 | 1990 | <100 |
| | | (1917-2163) | |
| +SEB | <100 | 290 | <100 |
| | | (164-416) | |
| +SED | <100 | 1500 | <100 |
| | | (1432-1568) | |
| +SEE | <100 | 2070 | <100 |
| | | (1910-2230) | |

95% confidence intervals in parenthesis.

TABLE 3
Cytokine production (pg/ml/10 T-cells) in GUT_{R}-2
after stimulation with superantigens (at PD 150).

| GUT_{R}-2 | TNFα | IFNγ | IL-10 |
|---|---|---|---|
| Complete | <100 | <100 | 2850 |
| medium | | | (2679-3021) |
| +SEA | <100 | <100 | 2840 |
| | | | (2738-2942) |
| +SEB | <100 | >25000 | >25000 |
| +SED | <100 | 470 | 5130 |
| | | (453-487) | (3899-5361) |
| +SEE | <100 | <100 | 5080 |
| | | | (4613-5867) |

95% confidence intervals in parenthesis.

### Example 3

Infliximab, a Chimeric Tnfα Antibody, Down-Regulates the INFγ Production in Activated Gut T-Lymphocytes in Crohn's Disease

Materials and Methods

Patients. The biopsy specimen were obtained from 5 patients with an established diagnosis of Crohn's disease according to clinical, radiological and histopathological criteria (1 male 22 years, and 4 females, mean: 38 years, range: 34-43 years). All the patients had active disease with a CDAI index above 150.

Biopsy specimens. Two colonic biopsies were obtained from each anatomical segment of the affected mucosa in each patient during colonoscopy (in total 16 biopsies). The biopsies were evaluated for histopathological changes. In each patient T-cells were cultured from four additional biopsies from mucosa with macroscopically active disease.

The study was approved by the local ethic committee of Aarhus County.

Cell culture. The four biopsies were washed twice in sterile PBS (saline) and once in the growth medium. The growth medium consisted of 90% RPMI 1640 10% human AB serum. 100 U/ml penicillin G 100 μg/ml streptomycin (basal medium, BM) supplemented with 2000 u/ml IL-2 and 500 u/ml IL-4 (complete medium). The T-lymphocytes were initially expanded in 5 ml complete medium and when cell density reached 1.5×10⁶/ml, the culture was split at a 1:2 ratio. From one female, two cultures were established from specimen taken 8 month apart (C1x and C11.3), and from the male two cultures were established from two different anatomical lesions (one from the cecum and one from the descending colon (C8.1 and C8.3 respectively). In the remaining three patients, one representative culture was used for the experiments. C1x, C2x and C4.2 are cultures grown for more than 150 days without further addition of antigen or feeder cells. C11.3, C12.1, C8.1, C8.3 are primary cultures cultured for less than 50 days.

Phenotyping and transmembrane TNFα. Monoclonal antibodies against CD3 (OKT3), CD4 (OKT4), CD8 (OKT8) and CD25 were obtained from hybridomas from American Type Culture Collection (ATCC). Monoclonal antibodies against CD45RO and HLA-DR were purchased from PharMingen. Monoclonal antibodies against TCR-1 (TCRα,β), TCR-2 (TCRγ,δ) and T cell receptor subfamily antibodies against V_{β}-chains were obtained through Coulter. An indirect immunofluorescence technique was applied to label the cells as previously described (ref. 12). 2×10⁴ events were analysed by flow cytometry (FACS Calibur, Becton Dickinson) and debris and aggregates were excluded by gating. Fluorescence microscopy was also applied to evaluate the staining. The antibody used for detection of transmembrane TNFα was obtained from R&D (FAB210 FITC). 5×10⁵ cells were obtained. 15 μl of undiluted antibody was added for 45 minutes. Unbound antibody was removed by washing, 2×10⁴ cells were analysed by flow cytometry (FACS). The binding of Infliximab was determined in an indirect way by a competitive assay with untreated cells as control.

In vivo activated primary cultures. These cells were washed once in RPMI 1640. They were then re-suspended in complete medium with and without Infliximab. Infliximab (obtained from Centocor, Malvern, Pa.) was added in a concentration of 5 μg/ml cell culture. Transmembrane TNFα and apoptosis was detected after one hour and 24 hours.

SEA stimulation of primary culture (C8.3) and cultures grown for more than 150 days (C1x,C2x,C4.2). Cells used had been cultured in complete medium. The cells were washed twice with RPMI 1640 in order to eliminate residual cytokines. They were then re-suspended in complete medium at a cell density of 10⁶/ml. Cells were then stimulated with Staphylococcus enterotoxins A (SEA) (obtained from Toxin Technology Madison, Wis.) at a concentration of 0.5 μg/ml cell culture. Two hours after stimulation cells were washed twice in RPMI 1640 and then re-suspended in complete medium with or without Infliximab (at a concentration as described previously). Transmembrane TNFα was determined one hour and 24 hours after the addition of Infliximab in activated cells and controls.

Cytokine determination. Supernatant of stimulated cells and controls was harvested after 24 hours. Cytokine matched antibody pairs for determination of IFNγ and tumour necrosis factor (TNFα) were obtained from Endogen. The detecting antibodies were all biotinylated. A time resolved fluorometric assay applying Europium labelled streptavidin and a Delphia 1234 fluorometer was used to determine the cytokine contents as described by the manufacturer (Wallac). Briefly, plates were covered with 50 μl of coating antibody at a concentration 2.5 μg/ml. They were placed at 4° C. overnight. Afterwards they were blocked with 10% AB-serum. Supernatant, controls and standards were added for two hours. Biotinylated antibody was added at a concentration of 1 μg/ml for one hour. Addition of Eu [symbol] marked streptavidin at a concentration 1:2000. Addition of enhancement solution. After 20 minutes, the plates could be read at a Delfia fluorometer. Because cytokine instability in low concentrations, new standards and dilutions were established for each determination. As the cell culture medium contained human serum, medium was controlled for cytokine content and levels were used as background. Concentrations below 30 pg/ml were not considered to be associated with cytokine producing T-cells. The data were analysed by a computer program (Assay Zap, Biosoft). Values were averages of three determinations.

Apoptosis and cytolysis. Annexin-FITC and propidium iodide were used for the determination of apoptosis (Nexins research and R&D). 5×10⁵ cells were obtained and placed in buffer for a half hour. Half of the cells were stained with 5 μl of Annexin-FITC diluted 1:10 in buffer, and 2.5 μl of propidium iodide. After incubation for 15 minutes the cells were analysed on a flow cytometer (FACS Calibur, Becton Dickinson). For the determination of cytolysis the same procedure was used after the addition of Infliximab and incubation with fresh human serum for one hour. A murine HLA class II antibody was used as a positive control for Infliximab.

Proliferation. Cell cultures were monitored with a Coulter counter measuring the increment in cell count after 24 hours. The channelysed count is measured on a 500 μl test sample. It was diluted 40 times in 20 ml Isoton II [symbol] (Coulter), so the cell count/ml was 80 times the channelysed count/ml.

Other methods. Cells were found to be free of mycoplasma by the Hoechst staining test.

Results

Cell culture and phenotype. When placed in the complete medium, lymphocytes migrated from the biopsy specimens and proliferation was evident within a week. After approximately two weeks the cell culture had expanded to more than 50×10⁶ cells. No antigen nor feeder cells were added. The in vivo activated T-cells were expanded only in the presence of high concentrations of IL-2 and IL-4. The phenotype of the primary cultures (C11.3, C12.1, C8.1, C8.3) is shown in FIG. 13 (representative example). Both TCR-1 and TCR-2 as well as CD4+ and CD8+ T cells that are present in situ are expanded in the cell culture medium. Upon continued culture a pure CD4+ cell line evolved within 40-50 days. C1x, C2x and C4.2 are representatives that have proliferated beyond 150 days. The cultures described above were used to study the effects of Infliximab on cytokine production, transmembrane TNFα, apoptosis, cytolysis and growth.

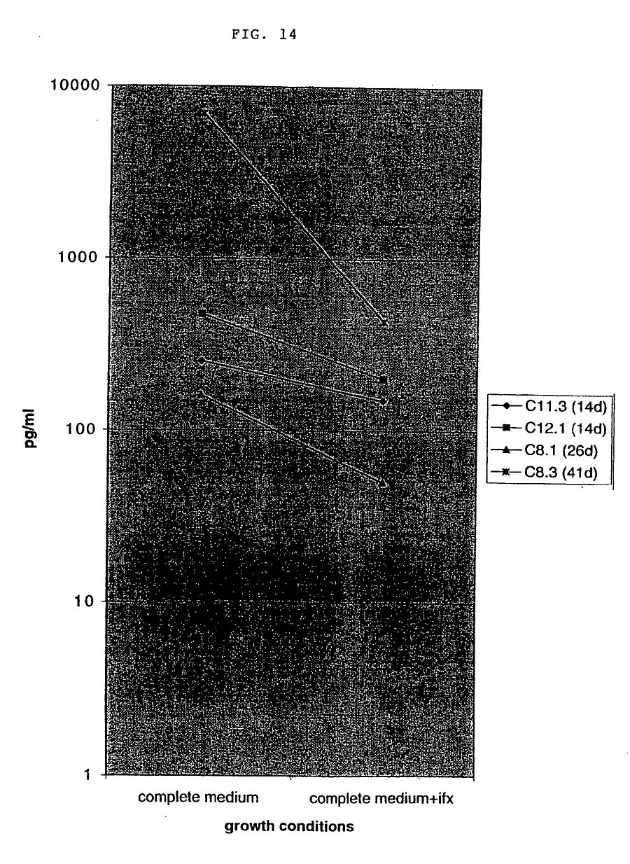

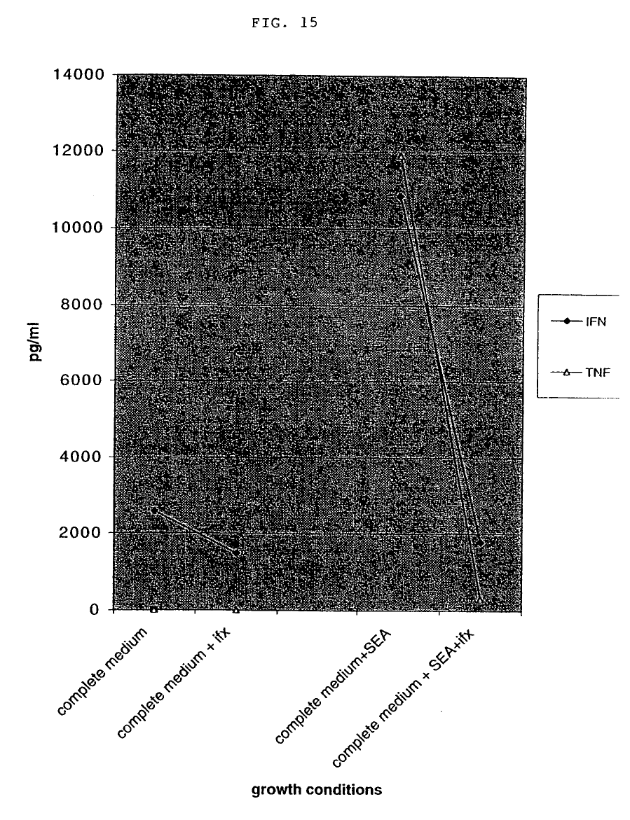

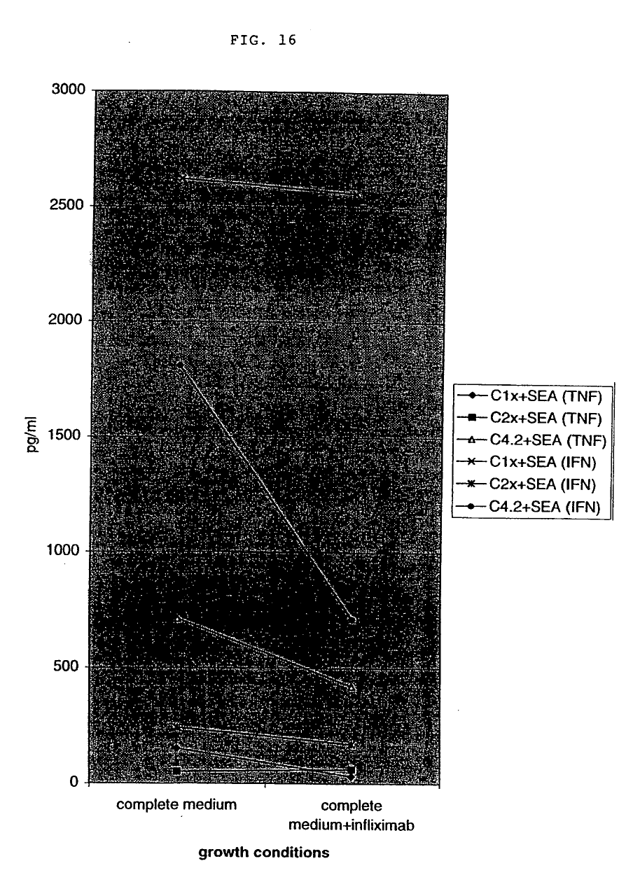

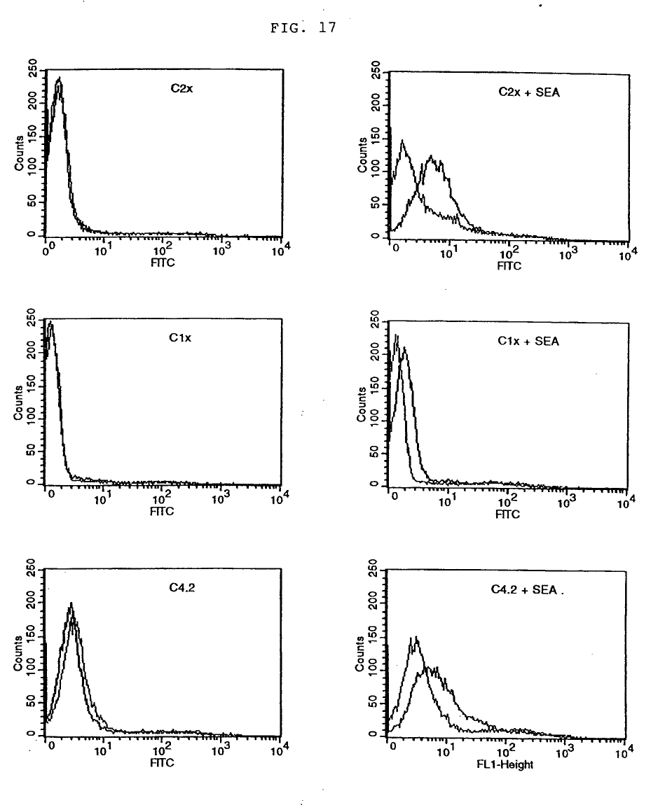

Cytokine production. In all the primary cultures a spontaneous production of IFNγ was observed. In all cultures, Infliximab induced a reduction in the 24 hour production of IFNγ (FIG. 14). As a control, recombinant IFNγ was added to the supernatant together with Infliximab (0.5 ng/ml recombinant IFNγ and 5 μg/ml Infliximab). The triple determination of IFNγ was 0.45 ng/ml. The TNFα productions in the primary cultures were close to detection level (<50 pg/ml), but this production was markedly enhanced by the stimulation with super-antigen (FIG. 15).

In the SEA stimulated primary culture, the effect of Infliximab on the absolute cytokine production was more pronounced (C8.1, 26 days, IFNγ: 25 to 12.81 ng/ml (49%), TNFα 1.95 to 0.05 (97%); C8.3, 35 days, IFNγ: 10.85 to 4.78 (56%) TNFα 11.9 to 0.3 (97%)) the reduction without stimulation with SEA was C8.1, IFNγ: 0.16 to 0.05 (68%), (TNFα below detection limit); C8.3: IFNγ: 2.58 to 1.47 (43%), (TNFα below detection limit).

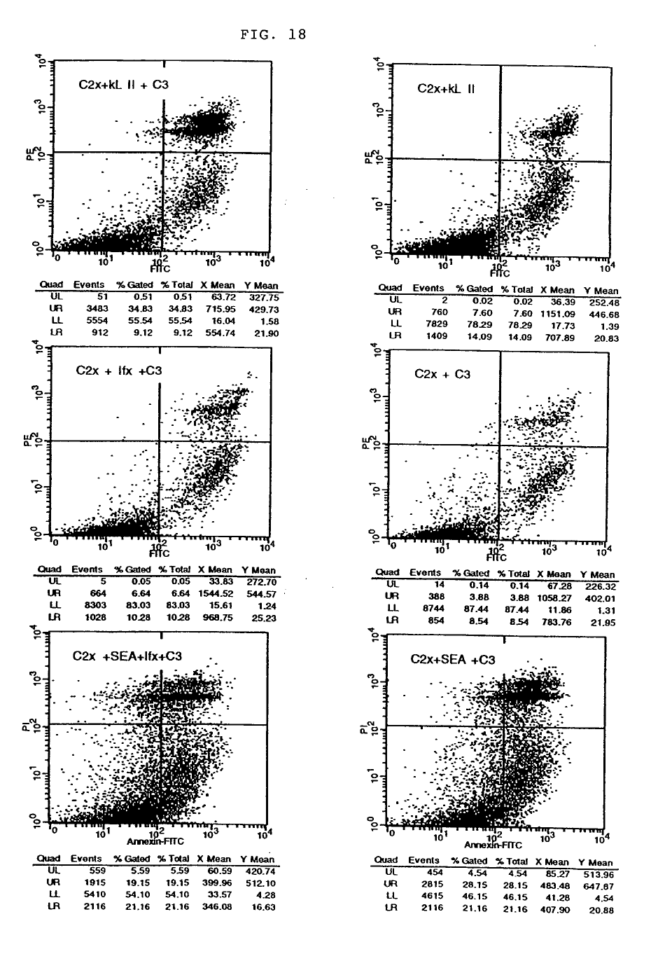

In the cultures grown for more than 150 days, there was not any constitutive production of IFNγ, but after stimulation with SEA an increase in the production of IFNγ and TNFα was observed. This cytokine production was also reduced by the addition of Infliximab (FIG. 16). There was no correlation between the level of IFNγ or TNFα production and the amount transmembrane TNFα.

Membrane bound TNFα and binding of Infliximab. The primary cultures all presents transmembrane TNFα determined by FACS analysis (FIG. 13). After addition of Infliximab to the cultures, the staining intensity of transmembrane TNFγ is reduced indicated by a left shift of the FACS curve. If these primary cultures were stimulated by super-antigen (SEA), an increase in the amount of transmembrane TNFα was observed, and the difference after supplement of Infliximab was more evident.

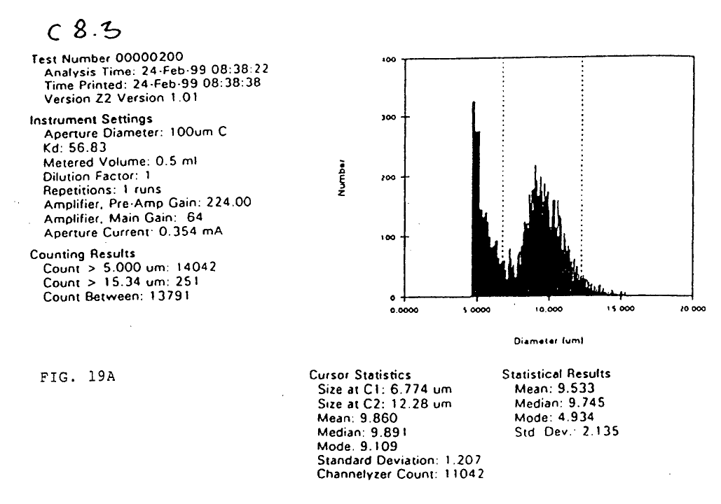

In the cell lines C2x, C1x and C4.2 transmembrane TNFγ was evident after stimulation with super-antigen SEA and Infliximab affected this relationship. No activation (indicated by transmembrane TNFα) could be demonstrated in these long term grown cultures before the addition of SEA (FIG. 17).

Apoptosis and cytolysis. FACS analysis of Annexin-FITC and propidium iodide stained cells was used as a measure of apoptosis and necrosis with and without the addition of complement. As a positive control a HLA-class II antibody was used.

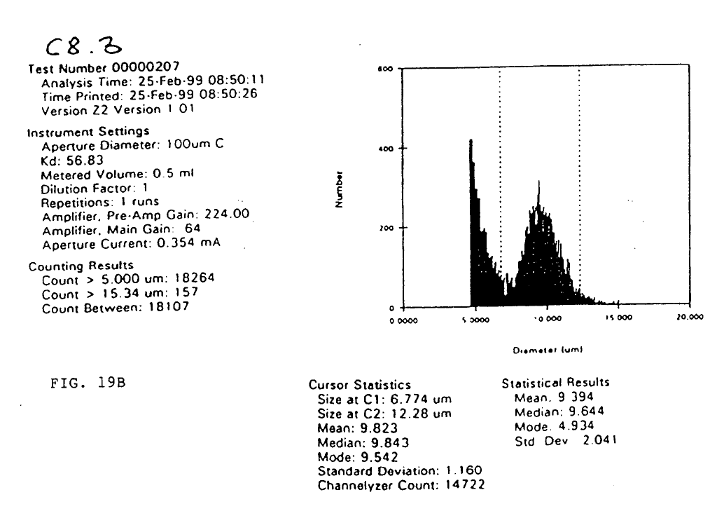

Infliximab did not induce any apoptosis in any of the in vivo activated primary cultures. In the long term cultured SEA stimulated C2x, Infliximab did not increase neither the amount of propidium iodide nor Annexin-FITC positive cells compared with SEA alone (FIG. 18) (HLA class II antibody as control).

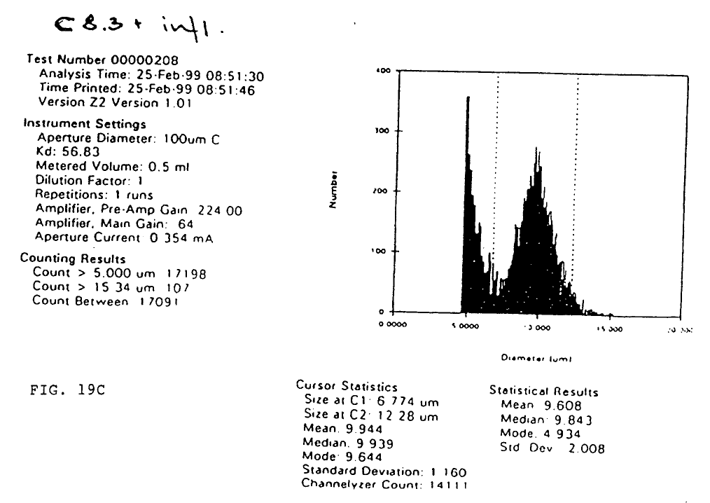

Proliferation. Proliferation was measured by a Coulter particle counter. Infliximab did not change the proliferation rate in any of the cultures (FIG. 19 A, B and C) (primary culture)). Cultures activated by SEA gave identical results.

Discussion. T-cell activation in Crohn's disease is one of the cornerstones in the inflammatory process with epithelial destruction (refs. 25, 26), probably because the production of pro-inflammatory Th1 cytokines INFγ and TNFα is increased (ref. 27).

Recent clinical studies in patients with Crohn's disease have demonstrated dramatic clinical responses following treatment with chimeric TNFα antibody (Infliximab) (refs. 7, 9). Different mechanisms have been proposed. Decreased production of TNFα by T-cells and the neutralisation of circulating TNFα may indirectly reduce the production of IFNγ (ref. 28).

Animal studies have demonstrated that transmembrane TNFα in the genetically engineered SP2/O myeloma cell line can bind Infliximab activating complement and macrophages resulting in cytolysis. In the present study, we have described the in vitro effects of Infliximab on in vivo activated T-cells obtained from the colon of patients with active Crohn's disease, regarding production of INFγ and TNFα, binding to transmembrane TNFα, apoptosis and proliferation. Infliximab down-regulates the IFNγ and TNFα production in all primary T-cell lines. These cultures revealed a spontaneous production of INFγ and to a lesser extend TNFα. This type 1 cytokine profile indicates that the primary cultures are in vivo activated since no antigen nor feeder cells has been added in vitro. In a previous study, it has been demonstrated that TNFα may be necessary for the LPMC production of IFNγ (ref. 28). Although not all the primary T-cell lines did produce detectable amounts of TNFα, Infliximab reduced the IFNγ production, probably by other mechanisms not involving TNFα synthesis. In cultures grown for more than 150 days, no residual in vivo derived antigen stimulation was present illustrated by the fact, that these cultures did not have any constitutive cytokine production. After SEA stimulation, a pro-inflammatory cytokine profile was present illustrated by increase in the production of INFγ and TNFα. Infliximab reduced the synthesis of both IFNγ and TNFα. No correlation was observed between the level of reduction in IFNγ and TNFα. Spontaneous or stimulated secretion of INFγ and TNFα in T-cells isolated from the mucosa of patients with Crohn's disease has been closely related to the degree of inflammation (refs. 9, 26, 28, 29), and the levels of TNFα secretion in pokeweed mitogen stimulated early cultures from patients with Crohn's disease in remission has also been related to the risk of relapse (ref. 30). The present in vitro data supports the clinical data. A decrease in disease activity in Infliximab treated patients would be expected if the INFγ and TNFα production is reduced in the activated intestinal T-cells.

Transmembrane TNFα is present in the primary cultures. The presence of transmembrane TNFα indicates a state of in vivo T-cell activation as illustrated previously (ref. 31). It has been shown that transmembrane TNFα correlates with the expression of the activation marker CD69. This finding is in good agreement with the Th1 cytokine profile in these cell lines. Only a minor fraction of the T-cells in the cultures are activated, but after addition of Infliximab, a left shift in the FACS curve is observed indicating the binding of Infliximab to the T-cells. If the culture is stimulated by super-antigen, the activation is more pronounced and the binding of Infliximab is demonstrated more clearly. In cultures grown for more than 150 days, no residual in vivo derived antigens are present. In these T-cell lines, no transmembrane TNFα could be demonstrated. After SEA stimulation, transmembrane TNFα was prominent, and a Infliximab-induced competitive inhibition could be shown. The 26 KD transmembrane TNFα is a co-stimulatory factor in the activation of B-cells (ref. 31). Inhibition of costimulatory signals by Infliximab binding to transmembrane TNFα may be of importance, and since only activated T-cells presents transmembrane TNFα, this effect may be confined to pro-inflammatory activated T-cells.

The Infliximab-induced reduction in cytokine production may be a result of a change in intracellular T-cell signalling either by a direct effect of binding to transmembrane TNFα or by an indirect effect because of changes in co-stimulation and T-cell interaction. Infliximab probably binds to other epitopes of transmembrane TNFα than the FAB 210F antibody. Substantial evidence is the fact that Infliximab neutralises circulating TNFα which FAB210F does not so simple correlation between the blocking effects can not be established.

In murine SP2/O myeloma cells, complement could be activated by binding Infliximab. This type of transmembrane TNFα was different from the wild-type by lacking two amino acids and a Ala in place instead of Val. This transmembrane TNFα was resistant to proteolytic cleavage. Scatchard analyses showed the cells of interest bound about 35000 Infliximab molecules per cell. We could not confirm these results in human T-cell lines. This might be related to proteolytic cleavage of human transmembrane TNFα or less extensive binding of Infliximab to in vivo activated human T-cells.

Apoptosis may be induced in response to various cytotoxic stimuli including activation of cell surface receptors such as Fas or TNFR1. The ligand for the transmembrane TNFα is not fully understood, but substantial evidence supports the hypothesis that the co-stimulatory signals are mediated by the p55 subunit (TNFR1) and not the p75 subunit (TNFRII) (ref. 31). We could not demonstrate any increased or decreased apoptosis by the binding of Infliximab to the transmembrane TNFα in any cultures.

Proliferation was unaffected in the cell lines ligated with Infliximab compared to the untreated cell lines. In clinical studies (ref. 28), patients responding to treatment with Infliximab disclosed reduced numbers of LPMC after a single dose.

In summary, we found that when activated T-cells binds Infliximab the production of the pro-inflammatory cytokines IFNγ and TNFα is reduced. Infliximab binds to transmembrane TNFα in activated human intestinal T-cells, and the binding is related to the level of activation demonstrated by FACS analysis and cytokine assays. We could not support results in murine myeloma cell lines where the binding of Infliximab activates complement resulting in cell lysis. Apoptosis and proliferation was unaffected by Infliximab. Changes in co-stimulatory signals via the TNFR-I might be a possible mechanism by which Infliximab exerts its effects.

### Example 4

Examples of T-Cell Vaccination

A. Multiple Sclerosis (MS)

1. A convenient amount, for example 50 ml, blood in heparin is drawn from a patient with MS.

2. The mononuclear cells of the blood that, other than lymphocytes, contain antigen presenting cells (APC) are isolated by a standard Ficoll-Isopaque gradient hydro-extracting.

3. The cells are disseminated in for example five culturing bottles in the medium consisting of 90% RPMI 1640, 10% human AB serum, antibiotic as well as 1000 u/ml IL-2 and 500 u/ml IL-4. If convenient other cytokines as GM-CSF and TNFα can be added to the bottles to increase/promote the maturing of the dendritic cells with a strong antigen presenting function.

At this stage, a selection for antigen activation (i.e. CD69+) may be included.

4. on day 0 antigen, in this case myelin, that the patient's auto-reactive T-cells react against, is added to one of the bottles. Instead of myelin components of the myelin can be added such as myelin basic protein or proteolipid protein or immune dominating epitopes deriving from these proteins.

5. This addition of antigen is repeated in the next bottle for example on day 2 and the procedure is continued with the other bottles with an interval of a couple of days.

6. Subsequently, the cells are propagated in the IL-2 and IL-4 containing medium. Notice that if “only” the life of the T-lymphocytes can be increased from 23 PD to 60 PD instead of 10′ cells one will have/get 2¹⁰-10¹⁸ cells, the equivalent of 1000 tons of cells, which will be sufficient to continue all further experiments and vaccination. In case the T-cells apparently does not have the expected ability for growth the antigen stimulation can be repeated, and furthermore co-stimulation with for example phorbolester or mitogen-stimulation may be tried to increase the growth potential.

7. The T-cells are tested for their antigen specificity and will after activating and attenuating (e.g. by γ-radiation, 60 Gy) be ready for T-cell vaccination.

8. Vaccination can be accomplished with 100-500×10⁶ T-cells in each forearm subcutaneously.

B. Insulin Dependent Diabetes

The same procedure as for A can be used, if only the antigen is for example glutamin acid decarboxylase (GAD)-65, GAD-67, insulin, or heat shock protein 60 (Hsp60)

C. Crohn's Disease and Ulcerative Colitis

Crohn's disease is a multifactorially conditioned chronic inflammatory intestinal disease where the normal tolerance of the immune system to the microbial intestinal flora is broken. Here the immune reactive T-cell clones (for T-cell vaccination) against the microbial flora can be brought about in the following way:

From a intestinal biopsy the aerob as well as the anaerob bacteria are cultured. After the culturing they are sonicated and can now be used as antigen/super-antigen. Subsequently, the biopsy is washed in a antibiotic-containing medium, and within 14 days the T-lymphocytes from the biopsy can be propagated in large number (>50×10⁶) in an IL-2 and IL-4-containing medium. Antigen presenting cells are obtained by ficoll separation of the patient's blood cells, and antigen specific/super-antigen specific continuous intestinal T-cell clones can now be propagated by adding antigen and γ-irradiated antigen presenting cells to the intestinal biopsy T-cells.

An analogous strategy can be used for patients with ulcerative colitis.

Note that for procedure A and B as well as for procedure C, the vaccination is individual (depending on the type of tissue), i.e. it has to be the patient's own cells that are used. Besides, note that T-cell vaccination primarily has been intended for persons that are already affected by diseases.

Activation (7 above) may be accomplished by mixing with a sonicated faeces sample from the patient. Such sample will contain the antigen that initially activated the T-cells in vivo. Therefore, the sonicate is suitable for boosting the T-cell lines prior to administration.

In a further alternative according to the invention in general, the biopsy or cell sample is cultured comprising IL-2 and IL-4 to enrich for activated T-cells, and the activated T-cells is isolated by immunomagnetic beads separation methods. The separated activated T-cells (which are often alloreactive) are then allostimulated and is further cultured in the presence the cytokines mentioned above. Hereby the alloactivated T-cells are expanded resulting in an antigen specific T-cell line.

This procedure may be used for any other relevant disease including the diseases mentioned above.

### Example 5

Establishment and Characterisation of In Vivo Activated T Cell Lines from Patients with Crohn's Disease in Preparation for Immune Therapy

Aim. T-cell vaccination (immunisation with attenuated auto-reactive T-cells) could be an attractive treatment option in patients with Crohn's disease. T-cell vaccination has not hitherto been possible, because auto-reactive T-cells have (like other human T-cells) limited replicative capacity in vitro (cellular senescence).

With the cell culture system described herein, it has been possible in certain situations, to expand and select in vivo activated T-cells in unlimited amounts. With this project we want to investigate whether such in vivo activated T-cells established from intestinal biopsies from patients with Crohn's Disease has reactivity against the patients own microflora, and if such T-cells could be used as a T-cell vaccination.

Background. Different studies has rendered that Crohn's disease is a multifactorial determined auto-immune disease where the normal tolerance against the microbial flora in the intestine is broken. The reactivity against the intestinal flora is mediated by reactive T-cells producing IFNγ and TNFα, and these cytokines contribute to the destruction of the intestinal mucosa (auto-immune reaction) in the diseased bowel. Treatment of Crohn's disease has lately been concentrated on interference with the immune response by using IL-10 or TNFα antibodies.

Animal experiments in murine models for auto-immune disease has demonstrated that immunisation with attenuated auto-antigen reactive T-cell clones (T-cell vaccination) was an effective treatment against these diseases. It has been hypothesised that the auto-reactive T cell clones, often with a Th-1 cytokine profile (producing IFNγ and TNFα) activates regulatory T-cells (IL-10 producing) in the immunological network. Regulatory T-cells are specifically directed against auto-reactive T-cells, and the production of IL-10 and TGFβ is immuno-suppressive to the auto-reactive cells and the bystander T-cells contributing in the auto-immune process. The advantage of T-cell vaccination to systemically treatment with IL-10 or TGFβ is that the regulatory T-cells are activated locally at the scene of inflammation and not associated with systemic adverse events. Besides, it is possible that T-cell vaccination activates other effector mechanisms in the immunological network, as e.g. cytotoxicity, against the auto-reactive T-cells.

In the murine experiments, auto-reactive T-cell lines used for vaccination have the advantage that they are continuous (immortal) resulting in unlimited amounts of T-cells available for the relevant studies. So far, it has been postulated that human T-lymphocytes are restricted by cellular senescence respecting the Hayflick limit (23±7 cell population doublings (PD)), one T-cell clone can expand to 2²³≈10⁷ T-lymphocytes. This amount is too little for a human T-cell vaccine.

Preliminary results. In certain situations, T-cells do not respect cellular senescence in vitro. We have shown that T-lymphocytes from patients with inflammatory skin diseases can be cultured continuously in a medium supplied with IL-2 and IL-4 but without antigen or accessory cells added (ref. 4, 12, 18). These immortal T-cell lines are activated in vivo in a way so they can be grown in vitro with unlimited replicative capacity. Recently a in vitro method has been demonstrated where T-lymphocytes can be immortalised in the presence of antigen and IL-2 and IL-4, cf. Example 1.

If the replicative capacity of T-cells can be increased from 30 PD to 50 PD, the amount of T-cells will increase from 2³⁰≈10⁹ cells (equivalent to 1 g cells) to 2⁵⁰≈10¹⁵ cells (1 ton cells). T-cell clones are usually expanded by using mitogen and radiated mononuclear cells or EBV immortalised B-lymphoblasts. None of these methods using feeder cell populations can immortalise human T lymphocytes, cf. Example 1.

Recently we have demonstrated that in vivo/in situ activated gut T-lymphocytes from patients with active Crohn's disease and with inducible INFγ production can be expanded in unlimited numbers (cf. Example 2). Such CD4+ T-cells expresses besides the T-cell receptor HLA Class-II antigens, and can auto-present super-antigens resulting in production of large amounts of IFNγ (cf. Example 2). These in vivo/in situ activated CD4+ T-cells with a type 1 cytokine profile are probably “auto-reactive” inflammatory T-lymphocytes. Preliminary results shows, that the inflammatory T-cells can activate regulatory CD4+ T-cells producing IL-10 indicating that the established inflammatory CD4+ T-cells could be used for T-cell auto-vaccination in patients with Crohn's disease.

Future Studies. We have established three inflammatory and corresponding three regulatory autologous continuous T-cell lines from gut biopsies of patients with Crohn's disease. The results are the substance in a protocol which probably can be used to develop in situ activated T-cells in unlimited amounts from most patients with Crohn's disease. One of the goals in the coming studies is to expand the inflammatory T-cells according to this protocol in a larger number of patients.

Activation of the inflammatory T-cells with antigens/super-antigens from the patients own intestinal flora will be of importance in the evaluation of the suitability of the cells as a T-cell vaccination. If they increase the production of type 1 cytokines after activation it indicates that the T-cells are auto-reactive. Antigen and super-antigen is obtained by cultures (aerobically and anaerobically) from rectal mucosa according to the methods described by Duchmann (ref. 6). As antigen presenting cells are used autologous PBMC or dendritic cells. It will be studied whether immune-modulating (immune down-regulating) drugs inhibits the pro-inflammatory response after activation antigen/super-antigen. Infliximab (chimeric TNFα antibody), 5-ASA, and steroids will be drugs of interest.

The interaction between autologous inflammatory and regulatory T-cells with and without externally activation will be analysed to describe if the type-/ideotype response has any implication in the activation of the regulatory T-cells when inflammatory T-cells are present.

Perspectives. In situ/in vivo activated T-lymphocytes from gut biopsies of patients with Crohn's Disease has a CD4+ phenotype and a cytokine profile (IFNγ) that is compatible with a “auto-reactive” origin. If it can be demonstrated that these continuos T-cell lines have reactivity against the patients own microflora, a T-cell/T-cell receptor peptide vaccination will be a potential option in these patients. If such a treatment has a positive effect, perhaps curative, it could be an option in other auto-immune diseases as multiple sclerosis and insulin dependent diabetes mellitus and inflammatory diseases as psoriasis, atopic dermatitis and rheumatoid arthritis.

The study has been approved by the Local Ethical committee of Aarhus County J. nr. 1997/3855, 1997/3856, 1998/4330, 1998/4419.

### Example 6

Cancer

Most cancers are associated with tumour infiltrating lymphocytes (TIL), and these TIL's are known to have killer cell activity against the tumour cells.

One example of cancerous diseases which could be treated with the T-cell lines or T-cells prepared according to the present invention is metastatic malignant melanoma.

The procedure could be as follows:

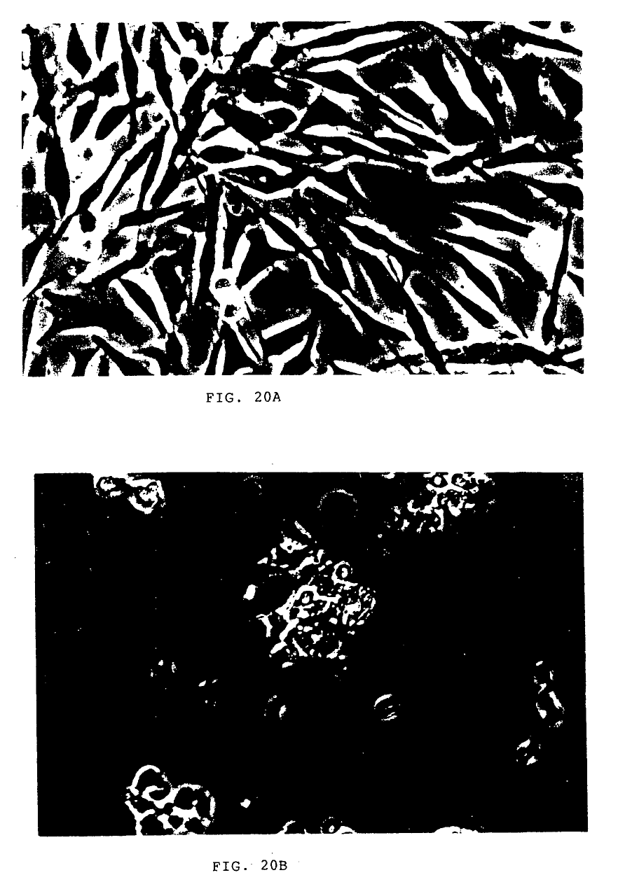

A cutaneous biopsy specimen or a lymph node biopsy specimen is known to harbour TIL's. The biopsy is divided into two, one part being cultured without cytokines in order to establish a tumour cell line (FIG. 20A). From the other part, T-lymphocytes are expanded in a medium supplemented with e.g. 10% human AB serum, 10 nM IL-2 and 2.5 nM IL-4 in the presence of 100 μM of the caspase inhibitor Z-VAD.

The outgrowing T-lymphocytes are in general of oligoclonal origin and consist of both CD4+ and CD8+ T-lymphocytes. Contained within the latter population are the presumed auto-immune effector cells (killer cells), while contained within the former population are CD4+ cells mediating help in generating CD8+ effector cells.

Following expansion, appropriate selection procedures may be used to select for CD8+ cells with tumour cell reactivity. FIG. 20B shows the result 24 hours after mixing an expanded CD8+ oligoclonal culture comprising cytotoxic cells with cytotoxic activity against autologous melanoma cells with melanoma cells. It should be noted that continuous T-cell lines are often oligoclonal for more than 100 PD, implying that continuous CD8+ tumour specific T-lymphocyte cell lines may react with several melanoma associated antigens, thus minimising the risk of tumour escape.

Selection for melanoma specific CD8+ cells may also be obtained by mixing outgrowing T-lymphocytes with tumour cells in a medium with IL-2, IL-4 and Z-VAD, because the tumour cells (target cells) acts as antigen presenting cells by directly presenting tumour associated peptides to CD0+ T-lymphocytes.

The tumour specific CD8+ T-lymphocytes are γ-irradiated in order to ensure that the cells cannot divide further and infused into the patient according to an established malignant melanoma IL-2 therapy protocol as already used by practitioners. Before administration, e.g. infusion, the T-lymphocytes can be incubated with the caspase inhibitor Z-VAD, in order to reduce AICD, or Z-VAD may be given during the administration.

Production of these cytokines together with IFNγ has consistently been found in 12 outgrowing T-lymphocyte cultures established from biopsies of patients with melanoma.

### REFERENCES

1. WO 88/07077 (The Children's Hospital, Inc.)
2. WO 94/02156 (The Board of Trustees of Leland Stanford Junior University)
3. WO 97/05239 (Celltherapy)
4. Kaltoft K et al., Cancer Genet. Cytogenet. 85, 68-71 (1995)
5. Effros R B, Pawelac P S, Exp. Cell Res. 25, 1129-1133 (1995)
6. Durchmann R et al., Clin. Exp. Immunol. 102, 448-455 (1995)
7. Dullemen H M et al., Gastroenterol. 109, 129-135 (1995)
8. Stronkhorst A et al., Scand. J. Gastroenterol. 27 Suppl., 61-65 (1992)
9. Targan S R et al., N. Engl. J. Med. 337, 1029-1035 (1997)
10. Groux et al.,
11. Gorrigan G J et al., Am. J. Resp. Cell Mol. Biol. 12, 567-578 (1995)
12. Kaltoft K et al., Arch. Dermatol. Res. 287, 42-47 (1994)
13. Rolfe F G et al., Am. J. Resp. Cell Mol. Biol. 17, 243-250 (1997)
14. Madaer R et al., Lancet 346, 807-808 (1995)
15. Zang J et al., Science 261, 1451-1454 (1993)
16. Rosenberg S A et al., JNCI 86, 1159-1166 (1994)
17. Kaltoft K et al., In Vitro Cell Dev. Biol. 28A, 161-167 (1992)
18. Kaltoft K et al., Cancer Genet. Cytogenet. 81, 13-16 (1995)
19. Perillo N L et al., Exp. Geront. 24, 177-187 (1989)
20. Kaltoft K et al., Arch. Dermatol. Res. 279, 293-298 (1997)
21. Christien J et al., Clin. Exp. Immunol. 73, 63-69 (1987)
22. Cottrez F et al., J. Immunol. Methods 172, 85-94 (1994)
23. Broux H, Nature 389, 737-742 (1997)
24. Pavelec et al., Immunological Review 16D, 31-42 (1997)
25. Lionetti P et al., Gastroenterol. 105, 373-381 (1993)
26. Pender S L et al., Gut 39, 284-290 (1996)
27. Deem et al., Clin. Exp. Imunol. 83, 79-84 (1991)
28. Plevy S E et al., J. Immunol. 159, 6276-6282 (1997)
29. Schreiber S L et al., Immunol. Today 13, 136-142 (1992)
30. Schreiber S L et al., Lancet 353, 459-461 (1999)
31. Aversa G et al., J. Exp. Med. 177, 1575-1585 (1993)

What is claimed is:

1. A feeder cell free cell culture system for generating a T cell line exceeding a life-span of at least 40 PD, wherein the cells in said T cell line are activated by a disease associated antigen, comprising
(a) T cells activated in vivo by a disease associated antigen;
(b) at least two factors which promote T-cell growth and
(c) one or more additional compounds promoting T-cell selection and expansion exceeding 40 PD.

2. The feeder cell free culture system of claim 1, wherein the factors which promote cytotoxic T-cell growth are cytokines which promote T-cell growth.

3. The feeder cell free culture system of claim 1, wherein the cytokines are selected from the group consisting of IL-2, IL-4, IL-7, IL-9, IL-10, IL-15, IL-16 and IL-12.

4. The feeder cell free culture system of claim 1, wherein a combination of at least one of IL-2 or IL-15 and at least one of IL-4 or IL-7 or IL-9 is used.

5. The feeder cell free culture system of claim 1, wherein a combination of IL-2 and IL-4 is used.

6. The feeder cell free culture system of claim 2, wherein each of the cytokines is used in a concentration of at least 1 nM.

7. The feeder cell free culture system of claim 2, wherein each of the cytokines is used in a concentration of at least 2.5 nM.

8. The feeder cell free culture system of claim 1, wherein the one or more additional compounds are selected from the group consisting of GM-CSF, caspase inhibitors, Z-VAD, alpha-CD95, IL-10, IL-12 and IL-16.

10. A continuous disease associated T cell line exceeding a life-span of at least 40 PD obtained by culturing the feeder cell free culture system of claim 1, wherein the disease associated T-cells in said T-cell line are associated with a disease of inflammatory, auto-immune, allergic, neoplastic or transplantation related origin, or combinations thereof.

11. An antigen free culture medium for culturing the feeder cell free culture system of claim 1 comprising
(a) two factors which promote T cell growth and
(b) one or more additional compounds promoting T cell selection and expansion.

12. A feeder cell free cell culture system for generating a T cell line exceeding a life-span of at least 40 PD, wherein cells in said T cell line are activated by a disease associated antigen comprising
(a) T cells activated in vivo by a non-skin disease associated antigen and
(b) at least two factors which promote T-cell growth.

13. A continuous T cell line exceeding a life-span of at least 40 PD, wherein said T cells are activated by a disease-associated antigen, wherein said antigen is associated with a disease of inflammatory or allergic origin selected from the group consisting of chronic inflammatory bowel disease, multiple sclerosis, type one diabetes rheumatoid arthritis, rejection-related disease and Graft-versus-host-related disease or wherein said antigen is associated with a disease of neoplastic origin selected from the group consisting of malignant melanoma, renal carcinoma, breast cancer, lung cancer, cancer of the uterus, prostatic cancer, hepatic carcinoma.

14. A method for obtaining a continuous associated T-cell line exceeding a life span of at least 40 PD comprising culturing the cell culture system of claim 1.

15. A method for obtaining the T-cell line of claim 1, wherein said cells are activated by a disease associated antigen having a life span exceeding at least about 40 PD comprising
(a) obtaining a tissue sample comprising T cells, activated by a disease-associated antigen, from a human patient, and
(b) culturing said tissue sample in the absence of feeder cells but in the presence of at least two factors which promote T-cell growth and one or more additional compounds promoting T cell selection and expansion exceeding 40 PD.

16. The method according to claim 15, wherein the tissue sample is selected from the group consiting of a biopsy, suptum, a swab, a gastric lavage, a bronchial lavage, an intestinal lavage and bodily fluid selected from the group consisting of spinal pleural, pericardial, synovial, blood and bone marrow body fluids.

17. A composition comprising the continuous T cell line of claims 10 and one or more excipients selected from the group consisting of water, saline, dextrose, glycerol, and ethanol.

18. A continuous melanoma specific CD8+ specific T cell line exceeding a life span of at least 40 PD obtained by the method comprising the steps of:
(a) obtaining a tissue sample from a cutaneous lymphoma of a patient
(b) culturing said tissue sample in the absence of feeder cells but in the presence of at least two factors which promote T-cell growth and one or more additional compounds promoting T cell selection and expansion exceeding 40 PD;
(c) selecting in said culture melanoma specific CD-8+ specific T cells.
